(19) United States
(12) Patent Application Publication
(10) Pub. No.: US 2007/0269563 A1
(43) Pub. Date: Nov. 22, 2007

(54) COMPOSITIONS AND METHODS FOR REDUCING MICROBIAL CONTAMINATION IN MEAT PROCESSING

(75) Inventors: Stephen P. Mixon, Montgomery, TX (US); Dennis M. Smithyman, Shreveport, LA (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(73) Assignee: Tasker Products, Inc.

(21) Appl. No.: 11/750,223

(22) Filed: May 17, 2007

Related U.S. Application Data
(63) Continuation-in-part of application No. 11/674,588, filed on Feb. 13, 2007.
(60) Provisional application No. 60/801,494, filed on May 17, 2006.

(51) Int. Cl. A23L 1/31; A61L 2/16

(52) U.S. Cl. 426/332; 422/292

(57) ABSTRACT

The present invention is directed to compositions, methods, and systems for reducing microbial contamination in meat processing. In accordance with one aspect of the invention, disinfection composition and/or recycled disinfection composition comprising an acid, a buffer, and optionally an antimicrobial metal is applying to a carcass during at least one processing step of sacrificing, scalding, feather/hair/hide removal, eviscerating, and washing. Other aspects of the invention provide a carcass processing system comprising processing stations intermittently fluidly connected via a buffered acidic disinfection composition.

### CROSS-REFERENCE TO RELATED APPLICATIONS

This application is a continuation-in-part of U.S. application Ser. No. 11/674,588, filed on Feb. 13, 2007, which in turn claims priority to U.S. Provisional Application Ser. No. 60/547,991, filed Feb. 26, 2004; each of which are incorporated herein by reference in their entirety. This application also claims priority from U.S. Provisional Application Ser. No. 60/801494, filed on May 17, 2006, incorporated herein by reference in its entirety.

### FIELD OF THE INVENTION

The present invention generally relates to reduction of pathogen load in meat processing.

### BACKGROUND OF THE INVENTION

Pathogen contamination of poultry carcasses is a major concern of chicken processors in the U.S. and the rest of the world. U.S. processors particularly face recent stringent governmental requirements related to maximal pathogen levels for meat and poultry slaughter facilities (see e.g., United States Department of Agriculture, Federal Register, 1996. 9 CFR Part 304 et al., Pathogen Reduction; Hazard Analysis and Critical Control Point (HACCP) Systems; Final Rule. Vol. 61, Number 144, pp. 38846-38848). New interventions will be required for processors to meet the new requirements.

Poultry is processed primarily to convert the bird's muscles into meat, to remove the unwanted components of the bird (blood, feathers, viscera, feet, and head), and to keep microbiological contamination at a minimum. The ultimate quality of the final product depends not only on the condition of the birds when they arrive at the plant, but also on how the bird is handled during processing. Various approaches have been utilized to lower pathogen prevalence on carcasses. The three main targeted areas for pathogen reduction include scald-tanks, rinse systems, and immersion chillers. For example, processors have increased water usage in rinses, scalders, and chillers from approximately 4-5 gallons per carcass to 7-10 gallons per carcass. Other processors have used a water recycling system that takes all rinse water from equipment sprays and carcass rinsers in the plant, and cleans the water using diatomaceous earth filters. The water is then ozonated, heated, and returned to the scalder.

Some processors have attempted to add sanitizers to rinse waters or common baths such as the scalder or chiller (see e.g., Okrend et al. (1986) J. Food Protect. 49, 500-503). The scalder is a common bath containing hot water in which the birds are submerged to soften feather follicles to aid in feather removal. The scalder is an area in which cross-contamination with Salmonella and other pathogenic bacteria is known to occur. But use of a sanitizer in the scalder to decrease cross-contamination has been difficult. Such a sanitizer must be resistant to binding with organic material, must not be driven off by high temperatures, and must have a relatively quick kill time. Those sanitizers currently known and used in the industry do not meet one or more of these requirements.

In poultry processing facilities throughout the U.S., rinse waters containing disinfectant are used throughout the plant. Locations where rinses are commonly used include: pre-scalding, picking, post-picking, post-evisceration, inside/outside bird washers (IOBW) prior to chilling, and automated reprocessing antimicrobial rinses prior to chilling. Additionally, most pieces of equipment with food contact surfaces are rinsed continually with water containing disinfectant.

The most commonly used disinfectant associated with equipment and carcass rinses in the U.S. is chlorine in the form of sodium hypochlorite (bleach). If the pH of the water is too high (>8.0), then bleach added to chlorinated rinse waters or chiller water will be ineffective, as the bleach will drive the pH up even further. At pH's above 8.0, chlorine is not found in its active form (hypochlorous acid) in high quantities and is ineffective for killing bacteria. Furthermore, if lye is used in the water reservoir that supplies the plant as a means of reducing the effects of acid rain, and the pipe that feeds the plant picks up this lye, then the pH of the water may be driven up to 10 or greater, causing problems with the chlorine as previously discussed. If ammonia has been added to the incoming water, there is a greater likelihood that when chlorine is added to the water, trichloramines will be formed, resulting in noxious odors.

Disinfectants approved for automated reprocessing of carcasses contaminated by fecal material include: trisodium phosphate (TSP), acidulated sodium chlorite (e.g., Sanova), peroxyacetic acid (e.g., Inspexx), and chlorine dioxide. But none of these disinfectants have been shown to be suitable for scalder use.

TSP is costly to use because of the high concentration (10%) used on carcasses. Residual TSP on carcasses causes the chiller water pH to increase dramatically. In plants where TSP is used, the chiller water will generally be in the pH range of 9.7 to 10.5. This is extremely high and prevents chlorine from being converted to its effective form. Often, use of TSP systems result in the increase of Salmonella prevalence when compared to levels prior to using TSP. To counter the effects of TSP-mediated pH increases, CO₂ gas systems have been added to the aeration systems of chillers as a means of reducing the pH so as to maintain the effectiveness of chlorine. It has also been reported that Listeria monocytogenes is resistant to the effects of trisodium phosphate (TSP). Furthermore, exposure to a high (8%) level of TSP for 10 minutes at room temperature is required to reduce bacterial numbers by 1 log 10 after a colony has grown on a surface and a protective layer (biofilm) has been formed.

Acidulated sodium chlorite (Sanova) is an approved poultry spray or dip at 500 to 1200 ppm singly or in combination with other generally regarded as safe (GRAS) acids to achieve a pH of 2.3 to 2.9 in automated reprocessing methods. In chiller water, sodium chlorite is limited to 50 to 150 ppm singly or in combination with other GRAS acids to achieve a pH of 2.8 to 3.2. Studies have shown that it can reduce Salmonella contamination from 31.6% prevalence to 10% prevalence (see e.g., Kemp et al. (2001) J. Food Protect. 64, 807-812). Many poultry processing facilities have switched from TSP systems to Sanova as an approved automated reprocessing system because it appears to reduce Salmonella more effectively than TSP.

Peroxyacetic acid (Inspexx), composed of hydrogen peroxide and acetic acid, has only recently been approved for use in the U.S. as an automated reprocessing disinfectant. Very little published research data exists regarding its efficacy in processing environments.

Some processors have attempted to reduce the temperature of one or more scald-tanks in an effort to increase yield (decreases the amount of fat cooked off of the carcass). For example, some processors have lowered the temperature of the first scald-tank to 108° F. (42.2° C.). While yield increases have been reported, pathogen count also greatly increases. At 108° F. (42.2° C.), most pathogens grow quite readily and they have all of the requirements for growth available (proper temperature, nutrients, pH, moisture, etc.). Thus, it is a general practice in the art that the temperature of the scalder should be maintained as high as possible without causing visible defects to finished carcasses, such as breast striping. Various scientific reports recommend no less than 10° F. higher than the maximum growth temperature of the pathogen. For example, Salmonella has a maximum growth temperature of 113° F. (45° C.). Thus, according to the conventional practice, the scalder should be no less than 123° F. (50.6° C.) in order to ensure that the pathogen cannot proliferate.

Processors have focused attention on bacterial reduction at the immersion chiller stage of processing (see e.g., Izat et al. (1989) J. Food Protect. 52, 670-673). Organic material in the chiller is primarily determined by the flow rate, flow direction, and the cleanliness of the scalder. The pH, temperature, flow rate, flow direction, chlorine concentration, and concentration of organic material (digesta, fat, blood) are all factors the pathogen-chiller load. The chlorine demand of an average chiller is very high at around 400 ppm because of the high content of organic material in the water. Because the maximum limit for chlorine use in the U.S. is only 50 ppm, there is rarely any true chlorine residual in the chiller overflow. Generally, the chiller pH is 6.5 to 7.5, the temperature below 40° F. (4.4° C.), the flow rate approximately ¾ to 1 gallon per bird), and the flow direction counter-current. The recycled chiller water can be ozonated and filtered to decrease organic material and to add an additional level of sanitation.

Thus there exists a need for methods to reduce pathogen load in meat processing plants, especially with regard to disinfectants compatible with high temperature, high organic load environments.

### SUMMARY OF THE INVENTION

The present invention is generally directed to methods to reduce pathogen contamination during food processing. The methods include the use of particular disinfection agents suited for processing of food products, preferably meat processing, and more preferably poultry processing, at or during one or more processing steps. These disinfection agents are generally non-oxidizing, acidic, buffered disinfectants that function efficiently in high temperature, high organic load, aqueous environments.

One aspect of the invention is directed to methods for reducing a microbial population on an animal carcass during processing including the steps of: (a) sacrificing an animal to form an animal carcass, (b) scalding an animal carcass, (c) removing feathers, hair, or hide from the scalded carcass, (d) eviscerating the defeathered, dehaired, or dehided carcass, (e) washing the eviscerated carcass, (f) chilling the eviscerated carcass, and (g) applying to the carcass during processing a disinfection composition including an acid and a buffer, in an amount and time sufficient to reduce a microbial population, wherein applying the disinfection composition (g) is performed during or immediately after at least one of steps (a), (b), (c), (d), (e), and (f). In accordance with a further embodiment, the methods can further include the steps of (h) recovering at least a portion of the applied disinfection composition, (i) adding to the recovered composition a sufficient amount of disinfection composition to yield a recycled disinfection composition, and (j) applying the recycled composition to a carcass during processing, wherein applying the recycled disinfection composition (j) is performed during or immediately after at least one of steps (a), (b), (c), (d), (e), and (f).

In various embodiments, the methods can include applying the disinfection composition and/or the recycled composition by submersing, rinsing, or spraying the carcass in or with the composition. In various embodiments, the methods can include applying the disinfection composition and/or the recycled composition at least by (1) submersing the carcass during scalding or (2) rinsing, spraying, or submersing the carcass after scalding but before eviscerating. In various embodiments, the methods above can include applying the disinfection composition or the recycled composition at least by (1) submersing the carcass during scalding, (2) rinsing, spraying, or submersing the carcass after scalding and before eviscerating, or (3) submersing the carcass during chilling.

In various embodiments, the methods can include rinsing, spraying, or submersing the carcass after scalding but before eviscerating is performed at a sanitization station, wherein the sanitization station is intermittently fluidly connected to (1) an apparatus for removing feathers, hair, or hide, (2) an apparatus for scalding, or (3) both an apparatus for removing feathers, hair, or hide and an apparatus for scalding, such that the recycled composition can be distributed from the sanitization station to the feather, hair, or hide removal apparatus, the scalder apparatus, or both the feather, hair, or hide removal apparatus and the scalder apparatus.

In various embodiments, the methods can include the application of a disinfection composition during submersion scalding, wherein the scalding occurs at a reduced temperature such that post-scalding carcass yield is increased. The scalding in at least one scalding tank can occur at a temperature from about 110° F. to less than about 123° F. In a further embodiment, scalding the animal carcass occurs in at least three scalding tanks; a first scalding tank operated at about 110° F. to about 120° F.; a second scalding tank operated at about 110° F. to about 125° F.; and a third scalding tank operated at about 120° F. to about 140° F.

In various embodiments, the disinfection composition and/or the recycled composition is of a pH from about 1.5 to about 4.0. In a further embodiment, the disinfection composition is of a pH of about 2 to about 3.

In various embodiments, the disinfection composition can include (1) sulfuric acid and (2) ammonium sulfate or sodium sulfate. In further embodiments, the disinfection composition can also include an antimicrobial metal. Such antimicrobial metal can be selected from copper, zinc, magnesium, silver, or iron. In various embodiments, the antimicrobial metal is copper in an active ionic form. In further embodiments, the disinfection composition has a copper concentration of about 1 ppm to about 20 ppm or is added to carcass processing water in an amount sufficient to provide a copper concentration of about 1 ppm to about 20 ppm. In further embodiments, the disinfection composition has a copper concentration of about 2 ppm to about 4 ppm or is added to carcass processing water in an amount sufficient to provide a copper concentration of about 2 ppm to about 4 ppm. In further embodiments, the disinfection composition has a copper concentration of about 3 ppm or is added to carcass processing water in an amount sufficient to provide a copper concentration of about 3 ppm.

In various embodiments, the disinfection composition can include (i) sulfuric acid, (ii) ammonium sulfate or sodium sulfate, and (iii) copper sulfate. In a further embodiment, the disinfection composition can include sulfuric acid, ammonium sulfate, and copper sulfate. In a further embodiment, the disinfection composition includes about 98% to about 99% water; about 0.1% to about 0.5% copper sulfate; about 0.1% to about 0.5% sulfuric acid; about 0.1% to about 0.5% ammonium sulfate; and has a pH of about 2 to about 3; a specific gravity at 25° C. of about 1.002; a boiling point of about 212° F.; and a freezing point of about 32° F.

In various embodiments, the disinfection composition can further include a stabilizing agent, wetting agent, hydrotrope, thickener, foaming agent, acidifier, pigment, dye, surfactant, or a combination thereof.

In various embodiments, the carcass is a poultry carcass, a beef carcass, or a pork carcass. In a further embodiment, the carcass is a poultry carcass. In a further embodiment, the poultry is a chicken, turkey, ostrich, game hen, squab, guinea fowl, pheasant, duck, goose, or emu carcass.

Another aspect of the invention is directed to a carcass processing system including a scalding station, a feather, hair, or hide removal station, and a sanitization station, wherein each station is intermittently fluidly connected via a buffered acidic disinfection composition. In various embodiments, the carcass can be a poultry, beef, or pork carcass. In a further embodiment, the carcass is a poultry carcass. In a further embodiment, the poultry is a chicken, turkey, ostrich, game hen, squab, guinea fowl, pheasant, duck, goose, or emu carcass. In various embodiments, the disinfection composition includes sulfuric acid; ammonium sulfate or sodium sulfate; and an antimicrobial metal. In further embodiments, the antimicrobial metal is copper in an active ionic form and the disinfection composition has a copper concentration of about 1 ppm to about 20 ppm or is added to carcass processing water in an amount sufficient to provide a copper concentration of about 1 ppm to about 20 ppm. In various embodiments, the carcass processing system includes at least three scalding tanks, wherein a first scalding tank is operated at about 110° F. to about 120° F.; a second scalding tank is operated at about 110° F. to about 125° F.; and a third scalding tank is operated at about 120° F. to about 140° F.

Other objects and features will be in part apparent and in part pointed out hereinafter.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 is a series of process flow diagrams. FIG. 1A depicts an overview of an animal carcass processing system. FIG. 1B depicts an alternative of a microorganism intervention system according to the present invention.

FIG. 2 is a process flow diagram depicting an alternative of a microorganism intervention system according to the present invention.

FIG. 3 is a process flow diagram for Tasker Blue® disinfectant agent application to poultry carcasses during processing as used in several studies. Further details as to methodology are provided in Example 1.

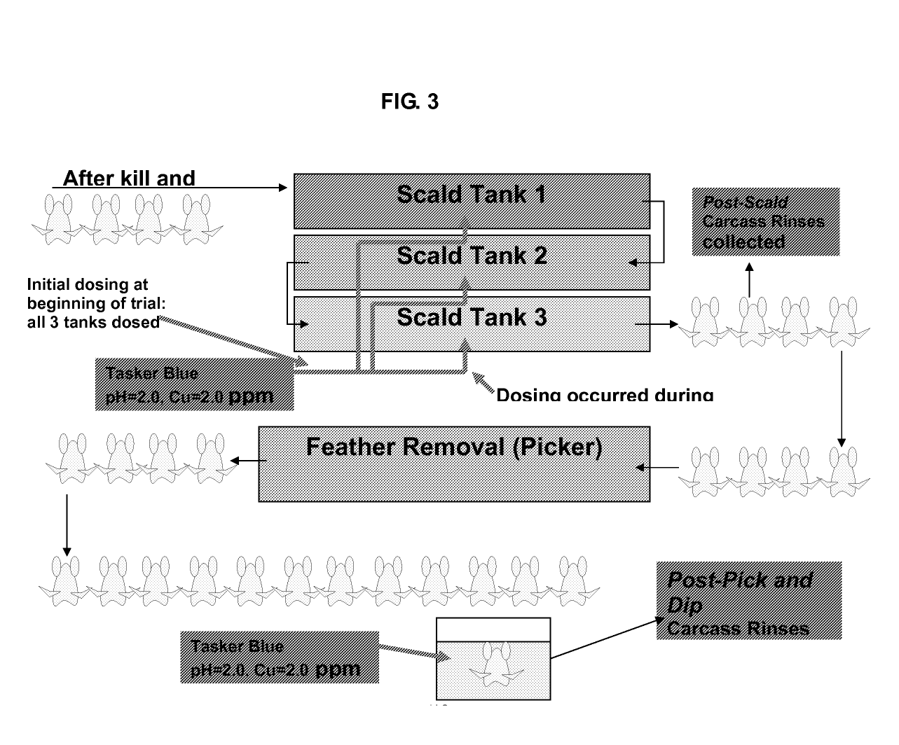

FIG. 4 is a bar graph showing the effect of the disinfectant Tasker Blue® on aerobic plate counts on broiler chicken carcasses during scalding. The log of colony forming units is shown over 13 days for control and treatment groups. An asterisk indicates a significant difference from control at p≦0.05. Additional details regarding methodology are presented in Example 1.

FIG. 5 is a bar graph showing the effect of the disinfectant Tasker Blue® on aerobic plate counts on broiler chicken carcasses after treatment in scalder and a post-pick dip solution. The log of colony forming units is shown over 13 days for control and treatment groups. An asterisk indicates a significant difference from control at p≦0.05. Additional details regarding methodology are presented in Example 1.

FIG. 6 is a bar graph showing the effect of the disinfectant Tasker Blue® on Escherichia coli counts on broiler chicken carcasses during scalding. The log of colony forming units is shown over 13 days for control and treatment groups. An asterisk indicates a significant difference from control at p≦0.05. Additional details regarding methodology are presented in Example 1.

FIG. 7 is a bar graph showing the effect of the disinfectant Tasker Blue® on Escherichia coli counts on broiler chicken carcasses after treatment in scalder and a post-pick dip solution. The log of colony forming units is shown over 13 days for control and treatment groups. An asterisk indicates a significant difference from control at p≦0.05. Additional details regarding methodology are presented in Example 1.

FIG. 8 is a bar graph showing the effect of the disinfectant Tasker Blue® on Salmonella prevalence on broiler chicken carcasses during scalding. The percent prevalence is shown over 13 days for control and treatment groups. A single asterisk indicates a significant difference from control at p≦0.05, while a double asterisk indicates a significant difference from control at p≦0.06. Additional details regarding methodology are presented in Example 1.

FIG. 9 is a bar graph showing the effect of the disinfectant Tasker Blue® on Salmonella prevalence on broiler chicken carcasses after treatment in scalder and a post-pick dip solution. The percent prevalence is shown over 13 days for control and treatment groups. A single asterisk indicates a significant difference from control at p≦0.05. Additional details regarding methodology are presented in Example 1.

FIG. 10 is a process flow diagram for the treatment group in studies examining Tasker Blue® disinfectant agent application to poultry carcasses at different scalder tank temperatures during processing as employed in Example 2. Further details as to methodology, including scalder tank temperatures, are provided in Example 2.

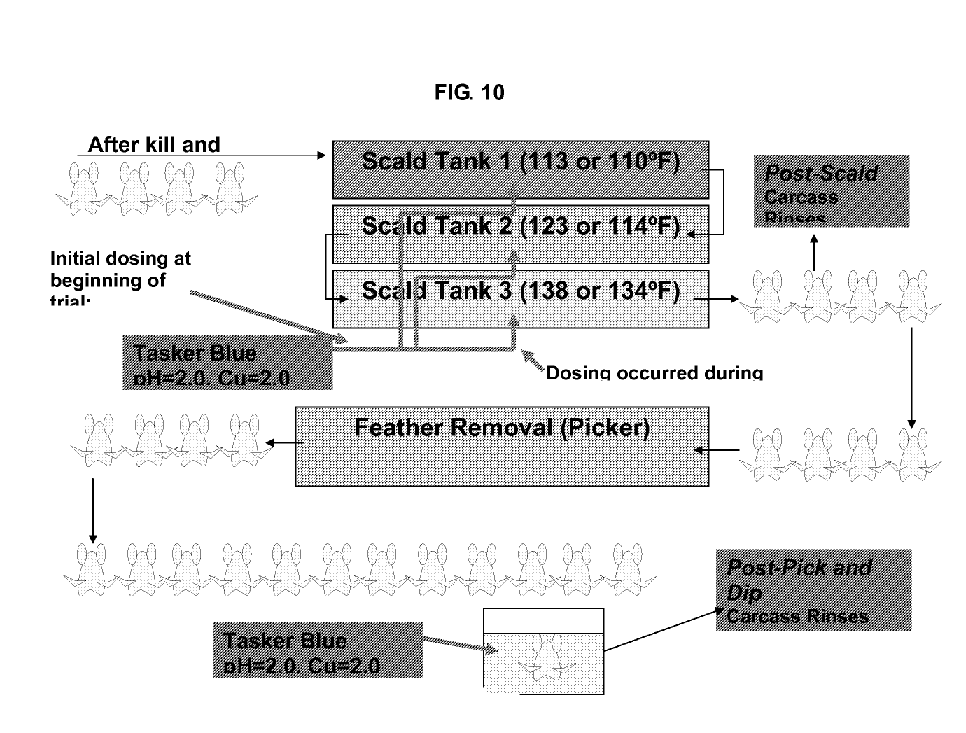

FIG. 11 is a process flow diagram for the control group in studies examining Tasker Blue® disinfectant agent application to poultry carcasses at different scalder tank temperatures during processing as employed in Example 2. Further details as to methodology, including scalder tank temperatures, are provided in Example 2.

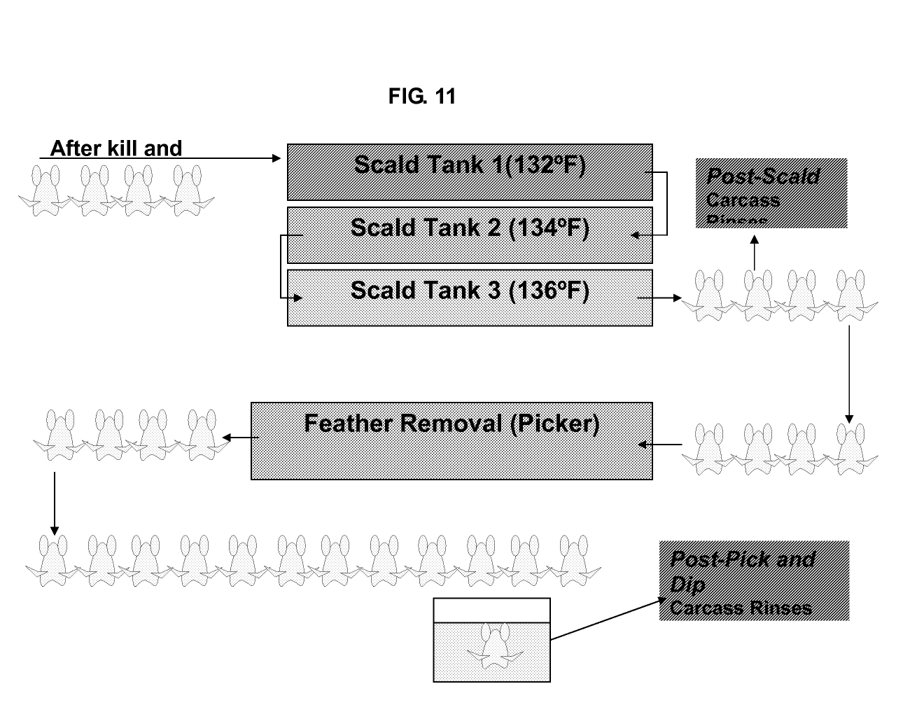

FIG. 12 is a process flow diagram of poultry processing depicting collection points in studies examining Tasker Blue® disinfectant agent application to poultry carcasses at different scalder tank temperatures during processing as employed in Example 2. Further details as to methodology, including scalder tank temperatures, are provided in Example 2.

FIG. 13 is a bar graph showing the broiler carcass yield (post-hoc cutter) at traditional scalding temperatures versus low scalding temperatures in combination with the disinfectant Tasker Blue®. The average weight (lbs) is shown for three replicates each of control and disinfectant treatment. Control scalder temperatures were 132°, 134°, and 136° F. for the first, second, and third scalder tanks, respectively. Scalder temperatures for the disinfectant treated tanks were 113°, 123°, and 138° F. for the first, second, and third scalder tanks, respectively. Mean values with different letters are significantly different at p<0.0001. Additional details regarding methodology are presented in Example 2.

FIG. 14 is a bar graph showing the broiler carcass yield (pre-IOBW) at traditional scalding temperatures versus low scalding temperatures in combination with the disinfectant Tasker Blue®. The average weight (lbs) is shown for three replicates each of control and disinfectant treatment. Control scalder temperatures were 132°, 134°, and 136° F. for the first, second, and third scalder tanks, respectively. Scalder temperatures for the disinfectant treated tanks were 110°, 114°, and 134° F. for the first, second, and third scalder tanks, respectively. Mean values with different letters are significantly different at p<0.0001. Additional details regarding methodology are presented in Example 2.

FIG. 15 is a bar graph showing the effect of Tasker Blue® applied during scalding, spraying, and chilling on Escherichia coli populations on broiler chicken carcasses. The number of colony forming units (log₁₀ units/mL) is shown over three replicates for control and disinfectant treated carcasses. Mean values with different letters are significantly different at p≦0.05). Additional details regarding methodology are presented in Example 3.

FIG. 16 is a bar graph showing the effect of Tasker Blue® applied during scalding, spraying, and chilling on Salmonella typhimurium prevalence on broiler chicken carcasses. The percentage of Salmonella positive chicken carcasses is shown over three replicates for control and disinfectant treated carcasses. Mean values with different letters are significantly different at p≦0.05). Additional details regarding methodology are presented in Example 3.

FIG. 17 is a process flow diagram depicting the impingement system for collecting air samples over poultry scalders treated with Tasker Blue® disinfecting agent. Further methodology information is provided in Example 4.

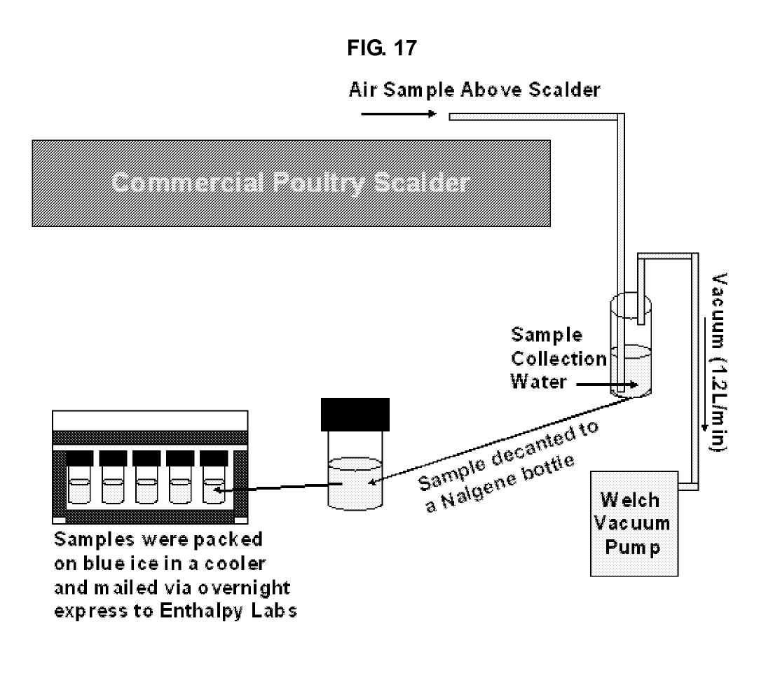

FIG. 18 is a bar graph showing the levels of ammonium sulfate, sulfuric acid, and copper sulfate in air collected above untreated (control) and treated scalder water containing Tasker Blue® disinfecting agent. Different letters within a particular chemical indicate a significant difference at p<0.0001. Further methodology information is provided in Example 4.

### DETAILED DESCRIPTION OF THE INVENTION

The present invention is generally directed to methods to reduce pathogen contamination during food processing. The methods include the use of particular disinfection agents suited for processing of food products, preferably meat processing, and more preferably poultry processing, at or during one or more processing steps.

Food Products

A food product generally includes any food substance that might require treatment with an antimicrobial agent or composition and that is edible with or without further preparation. Food products can include, for example, meat (e.g. red meat and pork), seafood, poultry, fruits and vegetables, eggs, egg products, ready to eat food, wheat, seeds, sprouts, seasonings, or a combination thereof. Red meat generally includes the meat of mammals such as beef, veal, mutton, lamb, rabbit, and horse. Produce generally includes food products such as fruits and vegetables and plants or plant-derived materials that are typically sold uncooked and, often, unpackaged, and that can sometimes be eaten raw.

The methods described herein can be applied to meat processing, especially poultry processing. A meat product generally includes various forms of animal flesh, including muscle, fat, organs, skin, bones, and body fluids and like components that form the animal. Animal flesh includes the flesh of mammals, birds, fishes, reptiles, amphibians, snails, clams, crustaceans, other edible species such as lobster, crab, etc., or other forms of seafood. The forms of animal flesh include, for example, the whole or part of animal flesh, alone or in combination with other ingredients. Typical forms include, for example, processed meats such as cured meats, sectioned and formed products, minced products, finely chopped products, ground meat and products including ground meat, whole products, and the like. For example, the methods of the present invention can be applied to processing of retail seafood. Such application provides odor knockdown and enhanced shelf-life.

Preferably, the methods described herein are applied to poultry processing. Poultry generally includes various forms of any bird kept, harvested, or domesticated for meat or eggs, and including chicken, turkey, ostrich, game hen, squab, guinea fowl, pheasant, quail, duck, goose, emu, or the like and the eggs of these birds. Poultry includes whole, sectioned, processed, cooked or raw poultry, and encompasses all forms of poultry flesh, by-products, and side products. The flesh of poultry includes muscle, fat, organs, skin, bones and body fluids and like components that form the animal. Forms of animal flesh include, for example, the whole or part of animal flesh, alone or in combination with other ingredients. Typical forms include, for example, processed poultry meat, such as cured poultry meat, sectioned and formed products, minced products, finely chopped products and whole products. Poultry processing methodology is well known in the art. Except as otherwise noted herein, therefore, the process of the present invention can be carried out in accordance with such processes.

Application

Food products can be contacted with the disinfection agent by any method or apparatus suitable for applying the disinfection agent. For example, the disinfection agent can be delivered as a vented densified fluid composition, a spray of the agent, by immersion in the agent, by foam or gel treating with the agent, or the like, or any combination thereof. Contact with a gas, a spray, a foam, a gel, or by immersion can be accomplished by a variety of methods known to those of skill in the art for applying agents to food.

The disinfection agents described herein can be employed for a variety of disinfection purposes, preferably as or for forming water-based systems for processing and/or washing animal carcasses. The present compositions and methods can be employed for processing meat at any step from gathering the live animals through packaging the final product. For example, the present compositions and methods can be employed for washing, rinsing, chilling, or scalding carcasses, carcass parts, or organs for reducing contamination of these items with spoilage/decay-causing bacteria, and pathogenic bacteria.

Carcass Processing

Before processing, live animals are generally transported to and gathered at the beginning of a processing line. Animals can be washed before entering the processing line. Processing typically begins with sacrificing the animal, typically by electrical stunning, followed by neck cutting and bleeding. A first washing step, known as scalding (e.g. submersion or immersion scalding) typically follows bleeding and loosens attachment of feathers, hair or hide of the animal. For example, poultry scalding loosens the attachment of feathers to the poultry skin. Submersion scalding can be accomplished according to the methods and employing compositions of the present invention. Submersion scalding typically includes immersing a stunned and bled animal (e.g., poultry) into a scalding hot bath of water or a liquid antimicrobial composition, typically at a temperature of about 50 to about 80° C., preferably about 50 to about 60° C. The liquid disinfection composition in the bath can be agitated, sonicated, or pumped to increase contact of the composition with the carcass. Scalding is generally conducted in a scald tank or trough, which contains the scalding liquid with sufficient liquid depth to completely submerse the poultry carcass. The carcass is generally transported through the tank or trough by conveyor at a speed that provides a few minutes in the scalding liquid.

According to the present invention, the scalding bath can include a disinfection composition described herein. The scalding bath can also include one or more of the additional ingredients permitted in scalding baths.

Inclusion of the disinfection solution in the scalding bath allows operation at a reduced temperature while still reducing pathogen contamination levels. Such reduction in temperature, allowed by the disinfection composition, provides a yield increase for post-scalder carcasses. In the absence of disinfection agent, operation of the scalder bath at lower temperature generally results in greatly increased pathogen prevalence and also inferior feather, hair, or hide removal. For example, in poultry processing, it is general practice to maintain the scalder a minimum of about 10° F. above the maximum growth temperature of Salmonella (113° F.). So, without using a disinfection composition, the lowest temperatures suggested in the art for the scalder bath is about 123° F. Addition of acidic buffered disinfection agents, described herein, allows the scalder baths to be maintained below this temperature while also providing reductions in pathogen contamination in the scalder water and on the emerging carcasses. For example, one or more poultry scalding bath can be maintained between about 110° F. and 123° F. Furthermore, scalder tanks operated at or near the maximum growth temperature of a microorganism can be used in combination with scalder tanks operated at conventional temperatures. For example, a sequence of three scalder tanks can be operated at about 110-120° F.; about 110-125° F. and about 120-140° F. respectively. Increases in yield resulting from lower scalder temperatures can include 0.5%, 1.0%, 1.5%, 2%, 2.5%, 3%, 3.5%, or 4% or more. Preferably, increases in yield resulting from lower scalder temperatures are 0.5% or greater.

After submersion scalding, the carcass is typically defeathered, dehaired, or dehided, and, optionally, singed before the next washing process. In the case of poultry processing, this second washing process is generally known as “dress” rinsing, “New York dress” rinsing, or post-pick rinsing, which rinses residual feathers and follicle residues from the carcass. Dress rinsing typically includes spraying a picked carcass with water, typically at a temperature of about 5 to about 30° C. To increase contact with the carcass, the disinfection compositions in the spray water can be applied at higher pressures, flow rates, temperatures, or with agitation or ultrasonic energy. Dress rinsing is typically accomplished with a washing apparatus such as a wash or spray cabinet with stationary or moving spray nozzles. Alternatively, a “flood”-rinsing or liquid submersion washing apparatus can be used immediately after picking.

According to the present invention, post-scalding rinsing (e.g., poultry dress rinsing) can be accomplished employing a disinfection composition described herein.

Dress rinsing is typically a final washing step before dismembering the carcass. Dismembering can include removing the head, the feet, eviscerating, and removing the neck, in any order commonly employed in carcass processing. The dismembered and eviscerated carcass can then be subjected to a washing step. In poultry processing, such washing step is known as inside-outside bird washing (IOBW). Inside-outside bird washing washes the interior (body cavity) and exterior of the bird. Inside-outside bird washing typically includes rinsing the interior and exterior surfaces of the carcass with streams or floods of water, typically at a temperature of about 5 to about 30° C. To increase contact with the carcass, the disinfection compositions in the spray water can be applied at higher pressures, flow rates, temperatures, or with agitation or ultrasonic energy. Inside-outside bird washing is generally accomplished by an apparatus that floods the bird carcass with streams of water in the inner cavity and over the exterior of the carcass. Such an apparatus can include a series of fixed spray nozzles to apply disinfection composition to the exterior of the bird and a rinse probe or bayonet that enters and applies antimicrobial composition to the body cavity.

According to the present invention, final washing (e.g., IOBW in poultry processing) can be accomplished employing a disinfection composition described herein.

After washing, both the interior and the exterior of the bird can be subjected to further decontamination. This further decontamination can be accomplished in part by a step commonly known as antimicrobial spray rinsing, sanitizing rinsing, or finishing rinsing. Such rinsing typically includes spraying the interior and exterior surfaces of the carcass with water, typically at a temperature of about 5 to about 30° C. To increase contact with the carcass, the disinfection compositions in the spray water can be applied using fixed or articulating nozzles, at higher pressures, flow rates, temperatures, with agitation or ultrasonic energy, or with rotary brushes. Spray rinsing is typically accomplished by an apparatus such as a spray cabinet with stationary or moving spray nozzles. The nozzles create a mist, vapor, or spray that contacts the carcass surfaces.

According to the present invention, antimicrobial spray rinsing, sanitizing rinsing, or finishing rinsing can be accomplished employing a disinfection composition described herein.

After spray rinsing, the bird can be made ready for packaging or for further processing by chilling, specifically submersion chilling or air chilling. Submersion chilling both washes and cools the bird to retain quality of the meat. Submersion chilling typically includes submersing the carcass completely in water or slush, typically at a temperature of less than about 5° C., until the temperature of the carcass approaches that of the water or slush. Chilling of the carcass can be accomplished by submersion in a single bath, or in two or more stages, each of a lower temperature. Water can be applied with agitation or ultrasonic energy to increase contact with the carcass. Submersion chilling is typically accomplished by an apparatus such as a tank containing the chilling liquid with sufficient liquid depth to completely submerse the poultry carcass. The carcass can be conveyed through the chiller by various mechanisms, such as an auger feed or a drag bottom conveyor. Submersion chilling can also be accomplished by tumbling the carcass in a chilled water cascade.

According to the present invention, submersion chilling can be accomplished employing a disinfection composition described herein.

Like submersion chilling, air chilling or cryogenic chilling cools the carcass to retain quality of the meat. Air cooling can be less effective for decontaminating the carcass, as the air typically would not dissolve, suspend, or wash away contaminants. Air chilling with a gas including an disinfection agent can, however, reduce the burden of microbial, and other, contaminants on the carcass. Air chilling typically includes enclosing the carcass in a chamber having a temperature below about 5° C. until the carcass is chilled. Air chilling can be accomplished by applying a cryogenic fluid or a gas or a refrigerated gas as a blanket or spray.

According to the present invention, air chilling can be accomplished employing a disinfection composition described herein. For example, air chilling compositions can include a gaseous or densified fluid disinfection composition.

After chilling, the carcass can be subjected to additional processing steps including weighing, quality grading, allocation, portioning, deboning, and the like. This further processing can also include methods or compositions according to the present invention for washing with disinfection compositions. For example, it can be advantageous to wash poultry portions, such as legs, breast quarters, wings, and the like, formed by portioning the bird. Such portioning forms or reveals new meat, skin, or bone surfaces which may be subject to contamination and benefit from treatment with a disinfection composition. Similarly, deboning a carcass or a portion of a carcass can expose additional areas of the meat or bone to microbial contamination. Washing the deboned carcass or portion with a disinfection composition described herein can advantageously reduce any such contamination. In addition, during any further processing, the deboned meat can also come into contact with microbes, for example, on contaminated surfaces. Washing the deboned meat with a disinfection composition can reduce such contamination. Washing can be accomplished by spraying, immersing, tumbling, or a combination thereof, or by applying a gaseous or densified fluid disinfection composition.

Usable side products of meat processing include heart, liver, and gizzard (e.g. giblets), neck, and the like. These are typically harvested later in processing, and are sold as food products. Of course, microbial contamination of such food products is undesirable. Thus, these side products can also be washed with a disinfection composition in methods of the present invention. Typically, these side products will be washed after harvesting from the carcass and before packaging. They can be washed by submersion or spraying, or transported in a flume including the disinfection composition. They can be contacted with a disinfection composition according to the invention in a giblet chiller or ice chiller.

The carcass, meat product, carcass portion, carcass side product, or the like can be packaged before sending it to more processing, to another processor, into commerce, or to the consumer. Any such product can be washed with a water based disinfection composition, which can then be removed (e.g., drained, blown, or blotted) from the poultry. In certain circumstances wetting the carcass before packaging is disadvantageous. In such circumstances, a gaseous or densified fluid form of the disinfection composition can be employed for reducing the microbial burden on the carcass. Such a gaseous composition can be employed in a variety of processes known for exposing a carcass to a gas before or during packaging, such as modified atmosphere packaging.

The advantageous stability of the disinfection compositions described herein in such methods, which include the presence of carcass debris or residue, makes these compositions competitive with cheaper, less stable, and potentially toxic chlorinated compounds. Preferred methods of the present invention include agitation or sonication of the use composition, particularly as a concentrate is added to water to make the use composition. Preferred methods include water systems that have some agitation, spraying, or other mixing of the solution. The carcass product can be contacted with the compositions described herein effective to result in a reduction significantly greater than is achieved by washing with water, or at least a 50% reduction, preferably at least a 90% reduction, more preferably at least a 99% reduction in the resident microbial preparation.

The present methods require a certain minimal contact time of the composition with food product for occurrence of significant disinfection effect. The contact time can vary with concentration of the use composition, method of applying the use composition, temperature of the use composition, amount of soil and/contamination on the food product, number of microorganisms on the food product, type of disinfection agent, or the like. Preferably the exposure time is at least about 5 to about 15 seconds.

Spraying

A preferred method for carcass washing employs a pressure spray of the disinfection composition. During application of the spray solution on the food product, the surface of the product can be moved with mechanical action, preferably agitated, rubbed, brushed, etc. Agitation can be by physical scrubbing of the meat product (e.g., poultry carcass), through the action of the spray solution under pressure, through sonication, or by other methods. Agitation increases the efficacy of the spray solution in killing micro-organisms, perhaps due to better exposure of the solution into the crevasses or small colonies containing the micro-organisms. The spray solution, before application, can also be heated to a temperature of about 15° to 60° C., preferably about 20° C., to increase efficacy.

Application of the material by spray can be accomplished using a manual spray wand application, an automatic spray of food product moving along a production line using multiple spray heads to ensure complete contact or other spray means. One preferred automatic spray application involves the use of a spray booth. The spray booth substantially confines the sprayed composition to within the parameter of the booth. For example, in poultry processing, the production line moves the poultry product through the entryway into the spray booth in which the poultry product is sprayed on all its exterior surfaces with sprays within the booth. After a complete coverage of the material and drainage of the material from the poultry product within the booth, the poultry product can then exit the booth in a fully treated form. The spray booth can include steam jets that can be used to apply the antimicrobial compositions of the invention. These steam jets can be used in combination with cooling water to ensure that the treatment reaching the poultry product surface is less than 65° C., preferably less than 60° C. The temperature of the spray on the poultry product is important to ensure that the poultry product is not substantially altered (cooked) by the temperature of the spray. The spray pattern can be virtually any useful spray pattern.

Immersing

Immersing a food product in a liquid disinfection composition can be accomplished by any of a variety of methods known to those of skill in the art. During processing of, for example, a poultry product, the poultry product can be immersed into a tank containing a quantity of washing solution containing disinfection agent. The washing solution is preferably agitated to increase the efficacy of the solution and the speed in which the solution reduces micro-organisms accompanying the food product. Agitation can be obtained by conventional methods, including ultrasonics, aeration by bubbling air through the solution, by mechanical methods, such as strainers, paddles, brushes, pump driven liquid jets, or by combinations of these methods. The disinfection agent can be heated to increase the efficacy of the solution in killing micro-organisms. After the food product has been immersed for a time sufficient for the desired effect, the food product can be removed from the bath or flume and the disinfection composition can be rinsed, drained, or evaporated off the food product. It is preferable that the poultry product be immersed in the washing solution after the poultry product have been eviscerated.

Foam Treating

In another alternative embodiment of the present invention, the food product can be treated with a foaming version of the disinfection composition. The foam can be prepared by mixing foaming surfactants with the disinfection agent or composition at time of use. The foaming surfactants can be nonionic, anionic, or cationic in nature. Examples of useful surfactant types include, but are not limited to the following: alcohol ethoxylates, alcohol ethoxylate carboxylate, amine oxides, alkyl sulfates, alkyl ether sulfate, sulfonates, quaternary ammonium compounds, alkyl sarcosines, betaines and alkyl amides. The foaming surfactant is typically mixed at time of use with the disinfection agent or composition. At time of use, compressed air can be injected into the mixture, then applied to the food product surface through a foam application device such as a tank foamer or an aspirated wall mounted roamer.

Gel Treating

In another alternative embodiment of the present invention, the food product can be treated with a thickened or gelled version of the disinfection agent. In the thickened or gelled state the disinfection agent remains in contact with the food product surface for longer periods of time, thus increasing the antimicrobial efficacy. The thickened or gelled solution will also adhere to vertical surfaces. The composition can be thickened or gelled using existing technologies such as: xanthan gum, polymeric thickeners, cellulose thickeners or the like. Rod micelle forming systems such as amine oxides and anionic counter ions could also be used. The thickeners or gel forming agents can be used either in the concentrated product or mixing with the disinfection agent, at time of use. Typical use levels of thickeners or gel agents range from about 100 ppm to about 10 wt-%.

Light Treating Poultry

In another alternative embodiment of the present invention, the food product can be exposed to an activating light (or other electromagnetic radiation) source following application of the disinfection agent. The activating light (or other electromagnetic radiation) can improve the efficacy of the disinfecting agent. The light can be ultraviolet light, infrared light, visible light, or a combination thereof. Other forms of electromagnetic radiation include radar and microwave.

Disinfection Agent

The disinfection agents utilized in the methods described herein are effective for killing one or more of the food-borne pathogenic bacteria associated with meat, particularly poultry, such as Salmonella typhimurium, Campylobacter jejuni, Listeria monocytogenes, and Escherichia coli, and the like. The disinfection compositions and methods of the present invention have activity against a wide variety of microorganisms such as Gram positive (for example, Listeria monocytogenes) and Gram negative (for example, Escherichia coli) bacteria, yeast, molds, bacterial spores, viruses, etc. The compositions and methods of the present invention, as described above, have activity against a wide variety of human pathogens. The compositions and methods can kill a wide variety of microbes on the surface of meat, e.g. poultry, or in water used for washing or processing of meat, e.g., poultry.

In several embodiments, reducing pathogen contamination in carcass processing is accomplished by using a non-oxidizing, acidic, buffered disinfectant that functions efficiently in high temperature, high organic load, aqueous environments. Preferably, the disinfectant operates at a low pH, for example around about pH 1 to about pH 4. More preferably, the disinfectant is a food safe additive (GRAS). Such a disinfectant can be utilized in several target steps of carcass processing, such as in scald-tanks, in rinse and/or dip systems, and in immersion chillers.

The preferred compositions include concentrate compositions and use compositions. Typically, a disinfection concentrate composition can be diluted, for example with water, to form a disinfection use composition. In a preferred embodiment, the concentrate composition is diluted into water employed for scalding, washing, chilling, or otherwise processing poultry.

Acidic Buffered and Acidic Buffered Copper Containing Disinfectants

Disinfectants within the scope of the invention include multiple-component disinfection agents. In one embodiment, the multiple-component disinfection agent is a buffered acidic disinfection agents. The disinfection agent can be a buffered acid solution of a strong acid and a salt of a strong acid and strong base (e.g., Tasker Clear). Exemplary acidic agents include those provided in Table 1. Exemplary buffering systems include corresponding salts.

For example, a buffered acidic disinfection agent for use in the methods described herein can be formed by reacting 98% sulfuric acid with a 26-28% by weight ammonium sulfate in water solution (order of addition is ammonium sulfate solution to sulfuric acid) at approximately 300-350° F. for 24 hours, where electrolysis of the reacting solution is applied for 1 hour at the start of the process, with a stabilization step (addition of more ammonium sulfate solution to ensure that the reaction is complete) after overnight cooling. As another example, the same process can be performed but at approximately 200-210° F. for 2 hours with a stabilization step immediately after the 1 hour electrolysis period. As a further example, a buffered acidic disinfection agent for use in the methods described herein can be formed, in a “cold process”, by adding 98% sulfuric acid slowly to a 30% by weight ammonium sulfate solution, with no stabilization step, at a temperature of 150-200° F. during the addition process. As yet another example, a buffered acidic disinfection agent for use in the methods described herein can be formed by reacting 8% sulfuric acid with a 26-28% by weight sodium sulfate in water (order of addition is sodium sulfate solution to sulfuric acid) for 4 hours at approximately 300-350° F. with a stabilization step (addition of more sodium sulfate solution to ensure that the reaction is complete) after cooling, where electrolysis of the reacting solution is applied for 1 hour at the start of the process. In still another example, a buffered acidic disinfection agent for use in the methods described herein can be formed, in a “cold process” (i.e., no electrolysis step), by reacting 98% sulfuric acid with a 26-28% by weight sodium sulfate in water solution for 4 hours at approximately 300-350° F. with a stabilization step after cooling.

In another embodiment, the multiple-component disinfection agent is a buffered acidic agent in combination with an antimicrobial metal-containing agent capable of providing free metal ions in solution. The multiple-component disinfection agent can be as described in, for example, U.S. Pat. No. 7,192,618; U.S. Patent Publication No. 2005/0191365 (U.S. applicatoin Ser. No. 11/065,678); and U.S. application Ser. No. 11/674,588, each of which are incorporated herein by reference. Examples of such antimicrobial metals include copper, zinc, magnesium, silver, and iron. Preferably, the multiple-component disinfection agent is a buffered acidic agent in combination with a copper containing agent capable of providing free copper ions in solution. Examples of various copper-containing agents include copper metal (inorganic copper), cuprous sulfate, cupric sulfate, and copper sulfate pentahydrate. The copper-containing buffered acidic disinfection agent for use in the methods described herein can be formed by the addition of various forms of copper to the various forms of acidic buffered disinfection composition described above.

In yet another embodiment, the multiple-component disinfection agent is an acidic agent in combination with a buffer, a sulfate-containing agent, and a copper-containing agent and capable of providing free copper ions in solution. In some embodiments, a single agent can deliver both copper ions and sulfate, for example copper sulfate. Such a mixture produces a copper sulfate complex that is highly protonated and at a low pH. Further, the sulfate component is thought to enhance copper and proton uptake by microbes. For example, a copper-containing buffered acidic disinfection agent, also containing sulfate, can be formed by mixing water (about 68%), one of the acidic buffered disinfection compositions described above (about 12%), and copper sulfate pentahydrate (about 20%) (e.g., Tasker Blue®). This low pH (buffered inorganic acidic) solution serves as the active (e.g., ionic Cu2+ form) carrier of copper.

The various copper-containing buffered acidic disinfection agents (e.g., Tasker Blue®) can be used in combination with additional buffered acidic disinfection agents (e.g., Tasker Clear) to achieve the prescribed pH control and copper content of the treatment solution. For example, the Tasker Clear product can be used for pH control, while the Tasker Blue® product can be used for copper control-these products can be added separately or in a pre-formulated blend of Clear® and Blue® to water to achieve the desired pH range (e.g., pH 1.5-3) and the desired copper range (e.g., 1-20 ppm). Water testing can be performed to determine the concentrations of Clear® and Blue® to add to achieve the desired targets.

Also preferable is that each of the disinfection agent ingredients are generally recognized as safe (GRAS) and are permitted for use as direct human food ingredients using good manufacturing practice.

Disinfectants described above can be produced in accord with the methods and formulations as described in U.S. patent application Ser. No. 10/922,604 (published as US 2005/0191394 A1); U.S. patent application Ser. No. 11/065,678 (published as US 2005/0191365 A1); U.S. Pat. No. 5,989,595; U.S. Pat. No. 6,242011B1; and U.S. Pat. No. 7,192,618, each of which are incorporated herein by reference. Generally, an effective acidic copper containing disinfectant agent can be made by combining an acid, a buffer, and a copper containing substance so as to reach a pH of about 1 to about 4 and a copper concentration of about 1 ppm to about 20 ppm, preferably about 3 ppm. For example, an acid, a buffer, and a copper containing substance can be combined in equal measure in a vessel at room temperature so as to reach a pH of about 2 and a copper concentration of about 3 ppm.

TABLE 1
Acids Generally Recognized as Safe (GRAS)

| Acid Name | CAS No. |
|---|---|
| ACETIC ACID | 000064-19-7 |
| ACONITIC ACID | 000499-12-7 |
| ADIPIC ACID | 000124-04-9 |
| ALGINIC ACID | 009005-32-7 |
| P-AMINOBENZOIC ACID | 000150-13-0 |
| AMINO TRI(METHYLENE PHOSPHONIC ACID), | 020592-85-2 |
| SODIUM SALT | |
| ANISIC ACID | 001335-08-6 |
| ASCORBIC ACID | 000050-81-7 |
| L-ASPARTIC ACID | 000056-84-8 |
| BENZOIC ACID | 000065-85-0 |
| N-BENZOYLANTHRANILIC ACID | 000579-93-1 |
| BORIC ACID | 010043-35-3 |
| (E)-2-BUTENOIC ACID | 003724-65-0 |
| BUTYRIC ACID | 000107-92-6 |
| CHOLIC ACID | 000081-25-4 |
| CINNAMIC ACID | 000621-82-9 |
| CITRIC ACID | 000077-92-9 |
| CYCLOHEXANEACETIC ACID | 005292-21-7 |
| CYCLOHEXANECARBOXYLIC ACID | 000098-89-5 |
| DECANOIC ACID | 000334-48-5 |
| 5-DECENOIC ACID | 085392-03-6 |
| 6-DECENOIC ACID | 085392-04-7 |
| 9-DECENOIC ACID | 014436-32-9 |
| (E)-2-DECENOIC ACID | 000334-49-6 |
| 4-DECENOIC ACID | 026303-90-2 |
| DEHYDROACETIC ACID | 000520-45-6 |
| DESOXYCHOLIC ACID | 000083-44-3 |
| 2,4-DIHYDROXYBENZOIC ACID | 000089-86-1 |
| 3,7-DIMETHYL-6-OCTENOIC ACID | 000502-47-6 |
| 2,4-DIMETHYL-2-PENTENOIC ACID | 066634-97-7 |
| ERYTHORBIC ACID | 000089-65-6 |
| 2-ETHYLBUTYRIC ACID | 000088-09-5 |
| 4-ETHYLOCTANOIC ACID | 016493-80-4 |
| FOLIC ACID | 000059-30-3 |
| FORMIC ACID | 000064-18-6 |
| FUMARIC ACID | 000110-17-8 |
| GERANIC ACID | 000459-80-3 |
| GIBBERELLIC ACID | 977136-81-4 |
| D-GLUCONIC ACID | 000526-95-4 |
| L-GLUTAMIC ACID | 000056-86-0 |
| GLUTAMIC ACID HYDROCHLORIDE | 000138-15-8 |
| GLYCOCHOLIC ACID | 000475-31-0 |
| HEPTANOIC ACID | 000111-14-8 |
| (E)-2-HEPTENOIC ACID | 018999-28-5 |
| HEXANOIC ACID | 000142-62-1 |
| TRANS-2-HEXENOIC ACID | 013419-69-7 |
| 3-HEXENOIC ACID | 004219-24-3 |
| HYDROCHLORIC ACID | 007647-01-0 |
| 4-HYDROXYBENZOIC ACID | 000099-96-7 |
| 4-HYDROXYBUTANOIC ACID LACTONE | 000096-48-0 |
| 4-HYDROXY-2-BUTENOIC ACID GAMMA-LACTONE | 000497-23-4 |
| 5-HYDROXY-2,4-DECADIENOIC ACID | 027593-23-3 |
| DELTA-LACTONE | |
| 5-HYDROXY-2-DECENOIC ACID DELTA-LACTONE | 051154-96-2 |
| 5-HYDROXY-7-DECENOIC ACID DELTA-LACTONE | 025524-95-2 |
| 4-HYDROXY-2,3-DIMETHYL-2,4-NONADIENOIC | 000774-64-1 |
| ACID GAMMA LACTONE | |
| 6-HYDROXY-3,7-DIMETHYLOCTANOIC ACID | 000499-54-7 |
| LACTONE | |
| (Z)-4-HYDROXY-6-DODECENOIC ACID LACTONE | 018679-18-0 |
| 5-HYDROXY-2-DODECENOIC ACID LACTONE | 016400-72-9 |
| 1-HYDROXYETHYLIDENE-1,1-DIPHOSPHONIC ACID | 002809-21-4 |
| 2-(2-HYDROXY-4-METHYL-3- | 057743-63-2 |
| CYCLOHEXENYL)PROPIONIC | |
| ACID GAMMA-LACTONE | |
| 4-HYDROXY-4-METHYL-7-CIS-DECANOIC ACID | 070851-61-5 |
| GAMMALACTONE | |
| 5-HYDROXY-4-METHYLHEXANOIC ACID DELTA- | 010413-18-0 |
| LACTONE | |
| 4-HYDROXY-4-METHYL-5-HEXENOIC ACID GAMMA | 001073-11-6 |
| LACTONE | |
| 4-HYDROXY-3-METHYLOCTANOIC ACID LACTONE | 039212-23-2 |
| HYDROXYNONANOIC ACID, DELTA-LACTONE | 003301-94-8 |
| 3-HYDROXY-2-OXOPROPIONIC ACID | 001113-60-6 |
| 4-HYDROXY-3-PENTENOIC ACID LACTONE | 000591-12-8 |
| 5-HYDROXYUNDECANOIC ACID LACTONE | 000710-04-3 |
| 5-HYDROXY-8-UNDECENOIC ACID DELTA- | 068959-28-4 |
| LACTONE | |
| ISOBUTYRIC ACID | 000079-31-2 |
| ISOVALERIC ACID | 000503-74-2 |
| ALPHA-KETOBUTYRIC ACID | 000600-18-0 |
| LACTIC ACID | 000050-21-5 |
| LAURIC ACID | 000143-07-7 |
| LEVULINIC ACID | 000123-76-2 |
| LIGNOSULFONIC ACID | 008062-15-5 |
| LINOLEIC ACID | 000060-33-3 |
| L-MALIC ACID | 000097-67-6 |
| MALIC ACID | 000617-48-1 |
| 2-MERCAPTOPROPIONIC ACID | 000079-42-5 |
| 2-METHOXYBENZOIC ACID | 000579-75-9 |
| 3-METHOXYBENZOIC ACID | 000586-38-9 |
| 4-METHOXYBENZOIC ACID | 000100-09-4 |
| TRANS-2-METHYL-2-BUTENOIC ACID | 000080-59-1 |
| 2-METHYLBUTYRIC ACID | 000116-53-0 |
| 3-METHYLCROTONIC ACID | 000541-47-9 |
| 2-METHYLHEPTANOIC ACID | 001188-02-9 |
| 2-METHYLHEXANOIC ACID | 004536-23-6 |
| 5-METHYLHEXANOIC ACID | 000628-46-6 |
| 4-METHYLNONANOIC ACID | 045019-28-1 |
| 4-METHYLOCTANOIC ACID | 054947-74-9 |
| 3-METHYL-2-OXOBUTANOIC ACID | 000759-05-7 |
| 3-METHYL-2-OXOPENTANOIC ACID | 001460-34-0 |
| 4-METHYL-2-OXOPENTANOIC ACID | 000816-66-0 |
| 3-METHYLPENTANOIC ACID | 000105-43-1 |
| 4-METHYLPENTANOIC ACID | 000646-07-1 |
| 2-METHYL-2-PENTENOIC ACID | 003142-72-1 |
| 2-METHYL-3-PENTENOIC ACID | 037674-63-8 |
| 2-METHYL-4-PENTENOIC ACID | 001575-74-2 |
| 4-METHYLPENT-2-ENOIC ACID | 010321-71-8 |
| 3-METHYL-3-PHENYL GLYCIDIC ACID | 000077-83-8 |
| ETHYL ESTER | |
| 4-(METHYLTHIO)-2-OXOBUTANOIC ACID | 000583-92-6 |
| 2-METHYLVALERIC ACID | 000097-61-0 |
| MYRISTIC ACID | 000544-63-8 |
| NONANOIC ACID | 000112-05-0 |
| (E)-2-NONENOIC ACID | 014812-03-4 |
| 2-NONENOIC ACID GAMMA-LACTONE | 021963-26-8 |
| 9,12-OCTADECADIENOIC ACID (48%) AND 9,12,15- | 977043-76-7 |
| OCTADECATRIENOIC ACID (52%) | |
| OCTANOIC ACID | 000124-07-2 |
| (E)-2-OCTENOIC ACID | 001871-67-6 |
| OLEIC ACID | 000112-80-1 |
| 3-OXODECANOIC ACID GLYCERIDE | 128331-45-3 |
| 3-OXODODECANOIC ACID GLYCERIDE | 128362-26-5 |
| 3-OXOHEXADECANOIC ACID GLYCERIDE | 128331-46-4 |
| 3-OXOHEXANOIC ACID DIGLYCERIDE | 977148-06-3 |
| 3-OXOOCTANOIC ACID GLYCERIDE | 128331-48-6 |
| 2-OXOPENTANEDIOIC ACID | 000328-50-7 |
| 2-OXO-3-PHENYLPROPIONIC ACID | 000156-06-9 |
| 3-OXOTETRADECANOIC ACID GLYCERIDE | 128331-49-7 |
| PALMITIC ACID | 000057-10-3 |
| 4-PENTENOIC ACID | 000591-80-0 |
| 2-PENTENOIC ACID | 013991-37-2 |
| PERACETIC ACID | 000079-21-0 |
| PERIODIC ACID | 010450-60-9 |
| PHENOXYACETIC ACID | 000122-59-8 |
| PHENYLACETIC ACID | 000103-82-2 |
| 3-PHENYLPROPIONIC ACID | 000501-52-0 |
| PHOSPHORIC ACID | 007664-38-2 |
| POLY(ACRYLIC ACID-CO-HYPOPHOSPHITE), | 071050-62-9 |
| SODIUM SALT | |
| POLYACRYLIC ACID, SODIUM SALT | 009003-04-7 |
| POLYMALEIC ACID | 026099-09-2 |
| POLYMALEIC ACID, SODIUM SALT | 030915-61-8 |
| POTASSIUM ACID PYROPHOSPHATE | 014691-84-0 |
| POTASSIUM ACID TARTRATE | 000868-14-4 |
| PROPIONIC ACID | 000079-09-4 |
| 2-(4-METHYL-2-HYDROXYPHENYL)PROPIONIC | 065817-24-5 |
| ACID-GAMMA-LACTONE | |
| PYROLIGNEOUS ACID | 008030-97-5 |
| PYRUVIC ACID | 000127-17-3 |
| SALICYLIC ACID | 000069-72-7 |
| SODIUM ACID PYROPHOSPHATE | 007758-16-9 |
| SODIUM BISULFATE (SODIUM ACID SULFATE) | |
| SORBIC ACID | 000110-44-1 |
| STEARIC ACID | 000057-11-4 |
| SUCCINIC ACID | 000110-15-6 |
| SULFAMIC ACID | 005329-14-6 |
| SULFURIC ACID | 007664-93-9 |
| SULFUROUS ACID | 007782-99-2 |
| TANNIC ACID | 001401-55-4 |
| TARTARIC ACID, L | 000087-69-4 |
| TAUROCHOLIC ACID | 000081-24-3 |
| 1,2,5,6-TETRAHYDROCUMINIC ACID | 056424-87-4 |
| THIOACETIC ACID | 000507-09-5 |
| THIODIPROPIONIC ACID | 000111-17-1 |
| TRIFLUOROMETHANE SULFONIC ACID | 001493-13-6 |
| (2,6,6-TRIMETHYL-2- | 015356-74-8 |
| HYDROXYCYCLOHEXYLIDENE)ACETIC ACID | |
| GAMMA-LACTONE | |
| UNDECANOIC ACID | 000112-37-8 |
| 10-UNDECENOIC ACID | 000112-38-9 |
| N-UNDECYLBENZENESULFONIC ACID | 050854-94-9 |
| VALERIC ACID | 000109-52-4 |
| VANILLIC ACID | 000121-34-6 |

Application Amounts of Acid/Sulfate Disinfection Agents

Generally, the longer the contact time with the meat surface, the higher the pH should be in order to minimize organoleptic damage. Conversely, shorter contact times allow a lower pH for better microbial reductions. For example, depending upon the contact time, the pH can be about 1.0, about 1.5, about 2.0, about 2.5, about 3.0, about 3.5, or about 4.0. Each application dosage is a function of effectiveness and cost. As the pH is a logarithmic scale, nearly 10 times more disinfectant is required to reach a pH of 2.0 as needed to reach a pH of 3.0.

Where the disinfection agent comprises an acidic buffered disinfection agent/composition, the actual application requirement is generally a function of the alkalinity of the processing plant water. The disinfectant is titrated until reaching the target pH, then monitored and maintained.

In those embodiments containing a buffered acid and an antimicrobial metal, the actual application requirement is generally a function of the desired target pH and the desired metal concentration. Preferably, the scalder, rinse, or bath solution contains an amount of added disinfection composition containing acid, buffer, and copper (e.g., Tasker Blue®) so as to reach a pH of about 1.5 to about 4.0 and a copper content of about 2 ppm to about 20 ppm. The pH can be adjusted independently by further addition of a disinfection composition containing acid and buffer (e.g., Tasker Clear).

Generally, the effectiveness of copper is highest at low pH; as the pH rises, the copper becomes bound and less effective. Preferably, the active, unbound copper concentration is about 2 ppm to about 4 ppm, more preferably about 3 ppm. To counter the risk of copper being bound, the disinfectant can be added up to about 20 ppm. Concentrations above this level should be avoided so as to minimize the risk of leaving residues on the carcasses. For example, depending on the pH, contact time, and the risk of copper being bound, the copper content of the scalder tank can be about 1 ppm, about 1.5 ppm, about 2 ppm, about 2.5 ppm, about 3 ppm, about 3.5 ppm, about 4 ppm, about 4.5 ppm, about 5 ppm, about 5.5 ppm, about 6 ppm, about 7 ppm, about 8 ppm, about 10 ppm, about 12 ppm, about 14 ppm, about 16 ppm, about 18 ppm, or about 20 ppm. For example, disinfectant can be added to the scalder water so as to reach a pH of about 2.0 and a copper content of about 3 ppm.

As a further example, poultry processing water containing disinfecting agent can about 98-99% water; about 0.1-0.5% copper sulfate, about 0.1-0.5% sulfuric acid; and about 0.1-0.5% ammonium sulfate. Such a wash can have a pH of about 2-3; a specific gravity at 25° C. of 1.002 or approximately 1.002; a boiling point of 212° F. or approximately 212° F.; and a freezing point of 32° F. or approximately 32° F.

The acidic buffered metal-containing disinfectants (especially the copper and sulfate containing formulations) are very effective at low concentrations and short exposure times. An effective killing Dose is usually measured as Concentration x Time (D=C×T). Generally, antimicrobial chemicals are used at concentrations in the 10's to 100's of ppm up to a full percentage range, and often for many minutes up to several hours, in order to be effective. The effective dose of the various above disinfectants for antimicrobial effect is much lower.

It is known that sulfate (SO₄²⁻), copper (Cu²⁺), and the ammonium ion (NH⁴⁺) are used by bacteria as part of their normal nutritional requirements. It is also known that at high concentrations copper sulfate can be used for plant disease control, as it is an effective antifungal agent, and will also control algae growth in lakes and ponds. The presence of sulfate, copper, and the low pH due to the presence of sulfuric acid provide the above disinfectant its antimicrobial properties.

The acidic buffered copper-containing disinfectant is non-oxidizing. This is in sharp contrast to other antimicrobial chemicals, such as chlorine compounds, ozone, and peracetic acid. Because it is non-oxidizing the disinfectant can be used in water based environments, such as the scald tank or chill tank used in poultry processing, where there is a significant amount of suspended or dissolved organic matter, without its effectiveness being impaired. Also, it will not produce oxidized compounds that will impart off-odors and flavors to the product or create toxic by-products such as tri-halomethanes (THM's). And, it will not cause the corrosion to plant and equipment typical of oxidizing chemicals.

Antimicrobial chemicals that are non-oxidizing are usually organic acids, such as lactic acid, or a combination of acids. Unlike the disinfectant containing sulfuric acid, ammonium sulfate, and copper sulfate, these organic acids are usually only effective at high concentrations, creating low pH environments where the organic acid molecules are in their undissociated, non-ionize state. In this form and concentration, the organic acid can pass through the cell membrane and gain entrance into the cell. Once inside the cell, the naturally higher pH of the cell will cause the acid to ionize and release protons (hydrogen ions). This will lower the internal pH of the cell. As cellular processes will only function optimally with the internal pH in a narrow range close to neutrality (pH 7), internal “proton pumps” are used to remove the unwanted protons from the cell. This process requires the use of energy (ATP). Bacterial cell growth therefore becomes inhibited due to a depletion of cellular ATP and reduced metabolic activity, as long as it remains in a low pH environment and in the presence of these organic acids.

While under no obligation to provide a mechanism of action, the following is the currently understood mechanism of action for the disinfectant containing sulfuric acid, ammonium sulfate, and copper sulfate pentahydrate. Such mechanistic explanation is not intended in any way to limit the invention described herein. It is known that sulfate is required for growth of the microbial cell. It provides the cell's requirement for sulfur for the formation of the sulfur containing amino acids cysteine, cystine, and methionine, which in turn are required for the synthesis of structural and enzymatic proteins. The bacteria have a well understood process that “actively transports” sulfate into the cell. Thus, in a complex environment bacterial cells will scavenge for sulfate in order to grow. The disinfectant containing buffered acid, sulfate, and copper exploits the sulfate ion scavenging function of bacterial cells.

The sulfate of the multi-component disinfectant is transported into the cell via the sulfate transport pathway and carries with it protons and copper ions. Once inside the cell, the protons are released and have to be removed via the energy consuming “proton pump.” In addition, the excess copper is now made available to bind to disulphide (—S—S—) and/or sulphydryl groups (—SH) associated with proteins. Interference with these groups can denature the proteins and destroy their structural or enzymatic activities, leading to the inhibition of cellular processes. Thus, there are several anti-microbial activities working in concert leading to the death of the cell: a depletion of ATP required for the removal of protons and the inactivation of structural and enzymatic proteins required for molecular synthesis.

Other Agents

In various embodiments, the method of the present invention employ a composition including peroxyacetic acid. Peroxyacetic (or peracetic) acid is a peroxycarboxylic acid having the formula: CH₃COOOH. Generally, peroxyacetic acid is a liquid having an acrid odor at higher concentrations and is freely soluble in water, alcohol, ether, and sulfuric acid. Peroxyacetic acid can be prepared through any number of methods known to those of skill in the art including preparation from acetaldehyde and oxygen in the presence of cobalt acetate. A solution of peroxyacetic acid can be obtained by combining acetic acid with hydrogen peroxide. A 50% solution of peroxyacetic acid can be obtained by combining acetic anhydride, hydrogen peroxide and sulfuric acid. Other methods of formulation of peroxyacetic acid include those disclosed in U.S. Pat. No. 2,833,813, which is incorporated herein by reference.

In some embodiments, the disinfection agent is any one or more of the multi-purpose acid compositions (e.g., a peroxyacetic acid-based disinfection agent) of U.S. Pat. No. 6,375,976, incorporated herein by reference. This disinfection agent is an acidic composition with a pH of less than 1, and is non-caustic to human tissue and safe for human ingestion. Such agent includes, for example, Inspexx® (Ecolab).

The peroxyacetic acid disinfection composition can be utilized in the various processing steps and systems described herein. For example, the peroxyacetic acid disinfection composition can be used in the scalder at a concentration of about 2 to about 50 ppm, preferably about 30 ppm. As another example, the peroxyacetic acid disinfection composition can be used in a dress rinsing at a concentration of about 50 to about 300 ppm, preferably about 200 ppm. As a further example, the peroxyacetic acid disinfection composition can be used in an inside-outside bird wash at a concentration of about 20 to about 200 ppm, preferably about 50 to about 100 ppm. As yet another example, the peroxyacetic acid disinfection composition can be used in a spray rinse at a concentration of about 50 to about 300 ppm, preferably about 100 to about 200 ppm. In a still further example, the peroxyacetic acid disinfection composition can be used in submersion chilling at a concentration of about 2 to about 100 ppm, preferably about 2 to about 30 ppm.

In another embodiment, the disinfection agent comprises phosphoric acid, hydrochloric acid, and citric acid (e.g., FreshFx®, SteriFx). For example, the Sterifx FreshFx® antimicrobial solutions comprise less than 5 wt % of each of phosphoric acid (CAS No. 7664-38-2), hydrochloric acid (CAS No. 7647-01-0), and citric acid (CAS No. 77-92-9). See e.g., Ingram et al, Southern Poultry Science Society Meeting Abstracts. Oct. 13, 2002, incorporated herein by reference in its entirety.

Amount Applied

In various embodiments, contacting the disinfection agent with the food product is accomplished with a quantity of antimicrobial agent sufficient to acceptably reduce the microbial burden in one or more stages of processing. In certain embodiments, contacting the disinfection agent with the food product at several stages of processing produces enhanced and/or synergistic reduction in microbial burden on the food product. The level of disinfection agent required for a desired effect can be determined by any of several methods. For example, food product samples can each be exposed to different amounts of disinfection agent. Then the food product samples can be evaluated for the amount of disinfection agent that yields the desired antimicrobial effect, and, preferably, for desired organoleptic qualities. The amount of disinfection agent required for antimicrobial effect at each processing stage can be reduced by application at several stages. Such a titration with disinfection agent can be conducted at several amounts of or treatment times in combination with treatment or exposure at other stages of processing, yielding a matrix of treatment results. Such a matrix yields a quantitative assessment of the amount of antimicrobial treatment required at various stages of processing to achieve a desired antimicrobial effect, and, optionally, desired organoleptic qualities. Synergy can be evaluated from such matrices using methods known to those of skill in the art.

The concentration of various disinfection agent can be as discussed above. Alternatively, the amount of disinfection agent added to scalder water can be the maximal amount approved by the Food and Drug Administration for a particular application, or some fraction thereof (e.g., about 50% -95%). As an example, the amount of disinfection agent added to scalder water is the 95%, 90%, 85%, 80%, 75%, 70%, 65%, 60%, 55%, or 50%, or less, of the maximal amount approved by the Food and Drug Administration for a particular application.

Processing Carcass Wash Water

Washing meat products can employ a large volume of water, or another carrier. Meat wash water can be used more than once (recycled), provided the water can be treated so that it does not transfer undesirable microbes to the meat being washed with the recycled wash water. One way to prevent the transfer of such undesirable microbes, is to reduce the microbial burden of the recycled wash water by adding one or more disinfection agents described herein. For example, if the fluid to be recycled is water-based and lacking any disinfection agent, a disinfection agent concentrate composition can be added to result in an effective antimicrobial concentration in the fluid to be recycled. Alternatively, if the fluid to be recycled already includes or has included a disinfection agent, a disinfection agent concentrate composition can be added to increase any concentration of disinfection agent to an effective antimicrobial level. It may be that the disinfection agent in the solution to be recycled has been totally depleted, in which case more of the disinfection agent composition is added.

In some circumstances, the water to be recycled includes a substantial burden of organic matter or microbes. If this is the case, the water may be unsuitable for direct recycling. However, if the water is to be recycled, a sufficient quantity of the disinfection agent composition can be added to provide an effective antimicrobial amount of the disinfection agent after a certain amount is consumed by the organic burden or microbes already present. Then, the recycled fluid can be used with disinfection effect. Routine testing can be employed for determining levels of disinfection agent, or of organic burden.

In the case of poultry processing, the method of recycling the poultry wash water includes recovering the poultry wash water, adding a composition of disinfection agent, and reusing the poultry wash water for washing poultry, for example, as described above. The poultry wash water can be recovered from steps in poultry processing including submersion scalding, dress rinsing, inside-outside bird washing, spray rinsing, and submersion chilling. Methods of recovering wash water from these steps are well-known to those skilled in the poultry washing and/or processing arts. The wash water can also be strained, filtered, diluted, or otherwise cleaned or processed during recycling. These steps can be modified for the corresponding steps for the processing of other meat products.

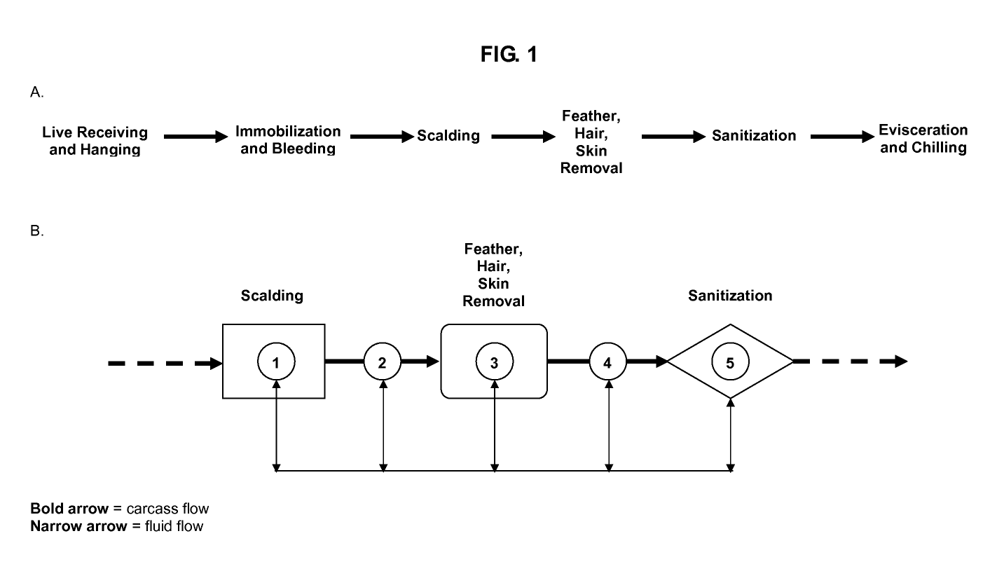

FIG. 1A provides an overview of an animal carcass processing system, the steps of which include:

Live Receiving and Hanging: Microorganism contamination is a concern at any of these steps. During live receiving and hanging, microorganism contamination can overload interventional controls in the system and can be carried forward to subsequent steps;

Immobilization and Bleeding: During immobilization and bleeding, voiding of feces can further contaminate the animal carcass which can further be carried forward to subsequent steps in the system;

Scalding: While the scalding process removes much of the dirt and feces contamination, microorganisms accumulate in the bath during multiple scalding processing steps. Thus, cross-contamination is increasing likely as multiple processes are completed;

Feather, Hair, Skin Removal: Similar to the scalding process, the apparatus which picks feathers, plucks hair, removes skin, and similar apparatus are implicated in cross-contamination of animal carcasses. Likewise, cross-contamination is increasingly likely as multiple processes are completed;

Sanitization: Some processes use a sanitization step to treat animal carcasses with a sanitizing agent (e.g., chlorine in a “New York” rinse in poultry) to provide some anti-microbial action; and

Evisceration and Chilling: Animal carcass rupture and spillage during the evisceration step contaminates the animal carcasses and equipment. Chilling, especially by immersion, is a cause of cross-contamination.

FIG. 1B depicts another alternative of a microorganism intervention system according to the present invention. Stations 1-5 are points where microbial intervention can occur both individually and in combination with other stations. An antimicrobial agent used in the Sanitization station (shown as station 5) can be reused in the Scalding station (station 1), the Feather Removal station (station 3) and intermediate stations (stations 2 and 4). An antimicrobial agent used at station 5 can also be reused at station 5. The bold arrows show the direction of animal carcass through the system. The narrow arrows show the flow direction of an antimicrobial agent through the system. Bidirectional arrows depict a flow direction which can be reversible or circular. Dashed arrows depict that the same or different processes can occur before station 1 and after station 5.

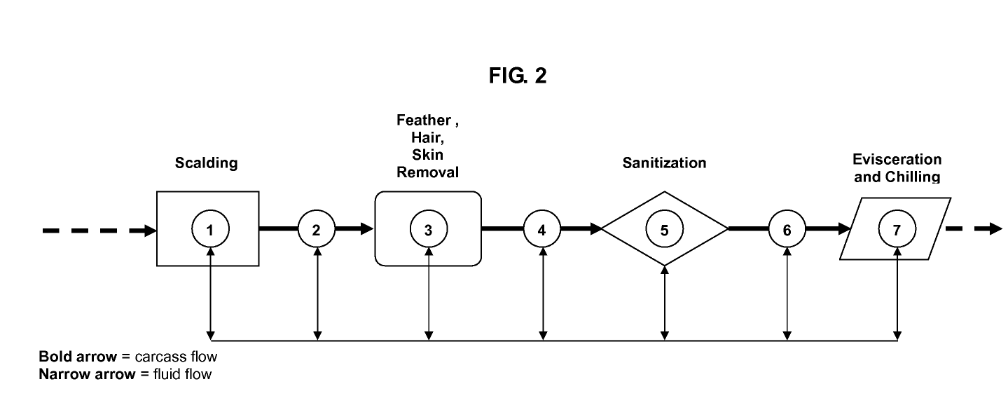

FIG. 2 depicts another alternative of a microorganism intervention system according to the present invention. Stations 1-7 are points where microbial intervention can occur both individually and in combination with other stations. An antimicrobial agent used in the Sanitization station (shown as station 5) can be reused in the Scalding station (station 1), the Feather Removal station (station 3), the Eviscerating station and the Chilling station (collectively shown as station 7) and intermediate stations (stations 2, 4 and 6). An antimicrobial agent used at station 5 can also be reused at station 5. The arrows show the flow direction of an antimicrobial agent through the system. Bidirectional arrows depict a flow direction which can be reversible or circular. Dashed arrows depict that the same or different processes can occur before station 1 and after station 7.

In one embodiment as depicted in FIG. 1B, the antimicrobial agent can be a liquid which can be applied by spraying on a carcass. Excess microbial agent can be removed from the carcass, e.g., by falling due to gravity, and the excess can be collected followed by distribution to stations 1-4 by suitable means, e.g., pumping. In an alternative, the excess microbial agent can be redistributed to station 5 which is sprayed on the same or another carcass. In certain embodiments where station 5 is enclosed or partially enclosed, the excess microbial agent can be collected through at least one opening in or near the bottom of station 5. The excess microbial agent can then be distributed to stations 1-4 by suitable means, e.g., pumping. In an alternative, the excess microbial agent can be redistributed to station 5 which is sprayed on the same or another carcass. In yet another embodiment, station 5 can be elevated above one or more of stations 1-4. Excess microbial agent can fall by gravity from the carcass directly onto one or more of stations 1-4. In another embodiment, station 5 can be elevated above one or more of stations 1-4, and the excess antimicrobial agent can be collected and distributed by gravity within an open or closed system, e.g. a gutter system, to one or more of stations 1-4. In another embodiment, the excess microbial agent can be collected and stored for a suitable period of time before distribution.

In an alternative embodiment as depicted in FIG. 2, the antimicrobial agent can be a liquid which can be applied by spraying on a carcass. Excess microbial agent can be removed from the carcass, e.g., by falling due to gravity, and the excess can be collected followed by distribution to stations 1-4, 6 and 7 by suitable means, e.g., pumping. In an alternative, the excess microbial agent can be redistributed to station 5 which is sprayed on the same or another carcass. In certain embodiments where station 5 is enclosed or partially enclosed, the excess microbial agent can be collected through at least one opening in or near the bottom of station 5. The excess microbial agent can then be distributed to stations 1-4, 6 and 7 by suitable means, e.g., pumping. In an alternative, the excess microbial agent can be redistributed to station 5 which is sprayed on the same or another carcass. In another embodiment, the excess microbial agent can be collected and stored for a suitable period of time before distribution. In yet another embodiment, station 5 can be elevated above one or more of stations 1-4, 6 and 7. Excess microbial agent can fall by gravity from the carcass directly onto one or more of stations 1-4, 6 and 7. In another embodiment, station 5 can be elevated above one or more of stations 1-4, 6 and 7, and the excess antimicrobial agent can be collected and distributed by gravity within an open or closed system, e.g. a gutter system, to one or more of stations 1-4, 6 and 7.

In addition to applying the antimicrobial agent to an animal carcass by spraying, the agent can be applied to the carcass by dipping, brushing, electrostatic spray, and any other suitable means whereby a portion of the agent remains on the carcass. In addition to removing the excess antimicrobial agent by gravity, the excess can additionally be removed by applying a centripetal force by rotating a carcass, by suction, e.g., applying a vacuum to a carcass, and any other suitable means. In each of the above embodiments, the excess microbial agent can be used with the same additional agent and/or mixed with a different antimicrobial agent or combination of antimicrobial agents. The pH and concentration of the solution applied to the carcass can be adjusted by methods known to those of skill in the art. Such adjustments can also be accomplished by automated detection and titration systems known to those of skill in the art. In addition, filters and other clarifying apparatus can be provided at individual or multiple stations within the system or in the distribution of the antimicrobial agent. Furthermore, stations 2, 4 and, in the case of FIG. 2, station 6 can comprise additional sanitizing means, e.g., pressurized liquid sprayers, which can emit the same or different antimicrobial agent or a liquid that does not contain an antimicrobial agent.

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

### EXAMPLES

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

The effect of using a scalder disinfectant was examined in a poultry scalder alone or in combination with a post-pick dip solution as a means of reducing pathogenic and indicator populations of bacteria on chicken carcasses. Sets of carcasses (10 per experimental group) were collected post-scald and post-pick and dip and evaluated for aerobic plate counts, E. coli counts, and Salmonella prevalence over a period of six weeks at a poultry processing plant.

For the scalder disinfectant treatment groups, all three scalders were initially dosed with pHarlo Blue® 0020 (Tasker) to a target level of 38 ppm (range: 30-60 ppm) with a target level of 0.8 ppm (range: 0.8-2.0 ppm) copper. pH was adjusted to a final pH of 2.0 to 2.2 and recorded. After the initial dose, the third scalder was continually dosed during the process with pHarlo Blue® 0020 to a target level of 38 ppm (range: 30-60 ppm) with a target level of 0.8 ppm (range: 0.8-2.0 ppm copper). The overflow water coming out of the scalder was monitored for pH and free copper and the level of material added to the incoming fresh water was adjusted based on these measurements.

Additionally, for the scalder and post-pick dip treatment group, a post-scald dip tank was used to treat post-pick carcasses. The dip solution was made by dosing tap water in a 44 gallon container to a pH of 2.0 and 2.0 ppm copper. Carcasses were removed from the line post-pick and dipped into the solution for 5-10 seconds. Ten carcasses were removed post scald and ten were removed post-pick and dip using the following technique to ensure that no bias was introduced.

Carcass selection was the same for all experimental groups. After at least 2 flocks had traversed the scalder, carcasses were removed post scald using the following technique to ensure that no bias was introduced. Carcasses were selected visually on the line post-scald, then the next five carcasses were counted aloud and the sixth carcass was selected for testing. The individual selecting the carcasses was wearing sterile examination gloves. In this way, no visual cues were used to introduce bias. Ten selected carcasses for each control and treatment were thus selected and were then hung on a sanitized rack, and allowed to drip. Sterile zip ties were used to cinch the neck of each chicken to prevent leakage of crop contents into the sample bag. Additionally, a sterile, unscented tampon was used to plug the vent of the chickens to prevent leakage of fecal material into the sample bag during shaking. In this way, the contents of the intestinal tract were not able to influence the microbiological effect of the disinfection ability of pHarlo Blue® 0020 during scalding. The carcasses were then individually bagged in sterile polyethylene bags and rinsed using 400 ml of sterile Butterfield's phosphate buffer by conducting the whole carcass rinse method as employed by USDA inspectors in processing facilities. The rinsate was encoded using a 4 digit number (to prevent identification by ABC employees and the introduction of bias) and sent on blue ice in a cooler using FedEx to ABC Research Corporation (Florida) for evaluation for APC, E. coli counts, and Salmonella prevalence.

These tests were conducted for 6 weeks and a total of 13 complete data sets were collected. Aerobic Plate Counts (APC) were determined using The Official Methods of Analysis of the AOAC, Method 990.12, and reported in colony forming units (CFU). E. coli were conducted using The Official Methods of Analysis of the AOAC, Method No. 990.12, and reported in colony forming units (CFU). Salmonella were tested using The Official Methods of Analysis of the AOAC, Method No. 2000.07, and reported as either positive or negative. Main effects of control versus treated were evaluated for each bacterial type. The overall experimental design was a 3×13×3×10 of treatment, day of collection, bacterial type evaluated, and chicken, for a total of 390 chickens and 1170 tests. Treatment effects were determined using t-tests and the Statistical Analytical Software (SAS) program for APC and E. coli counts. For Salmonella prevalence, Fishers Exact Test was conducted using SAS.

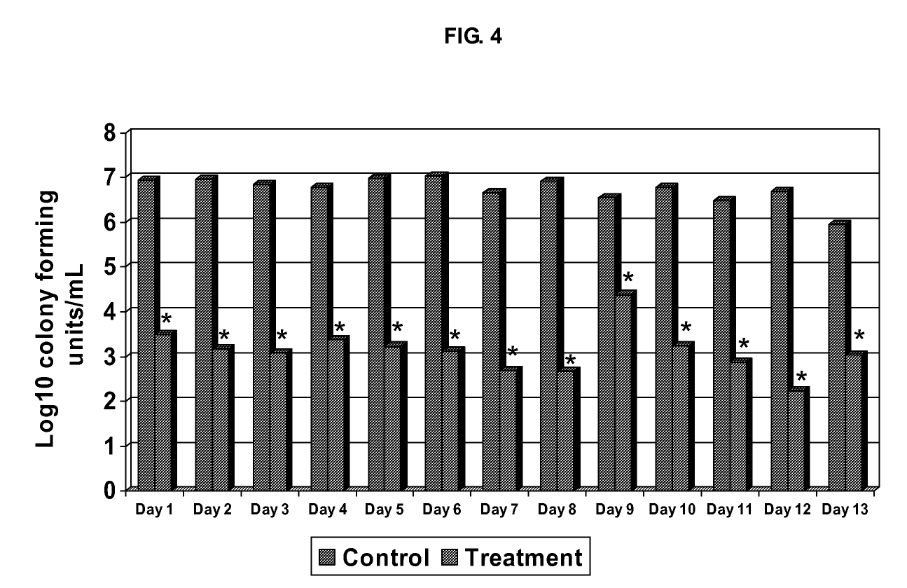

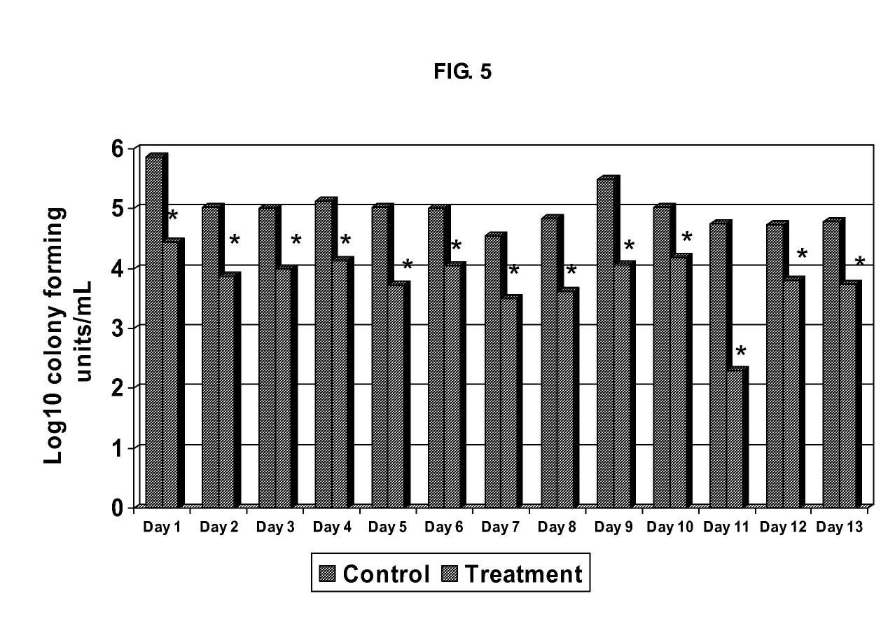

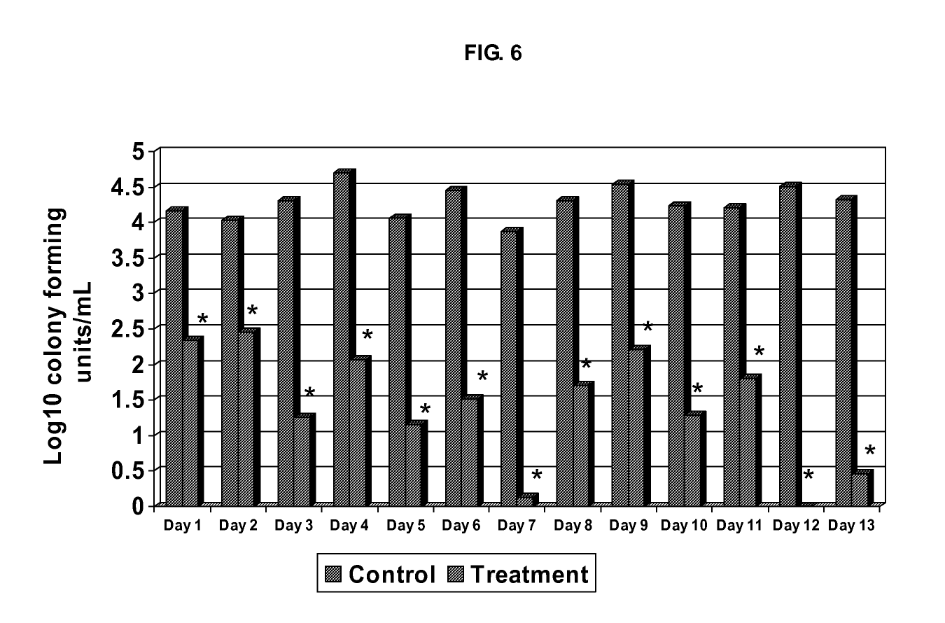

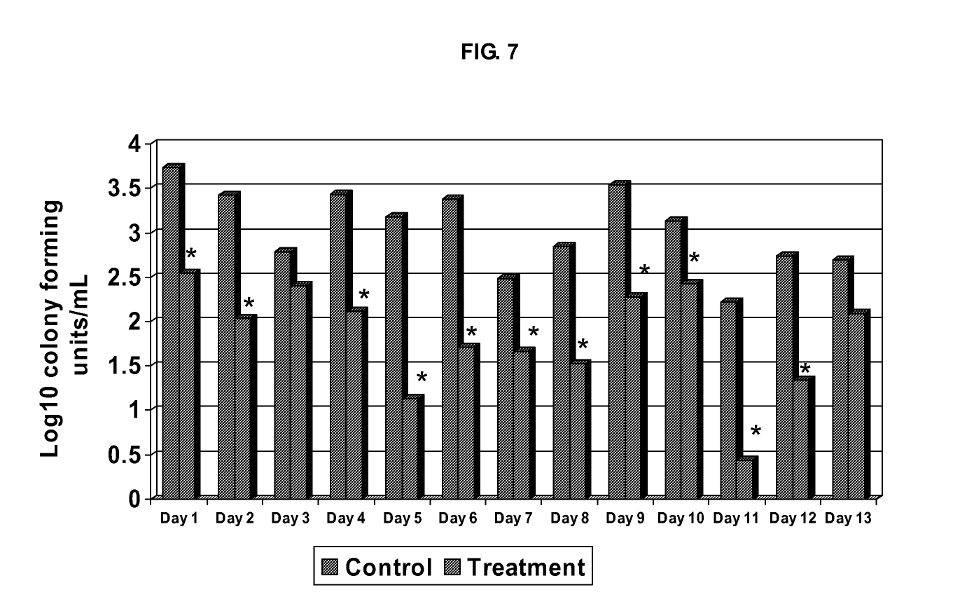

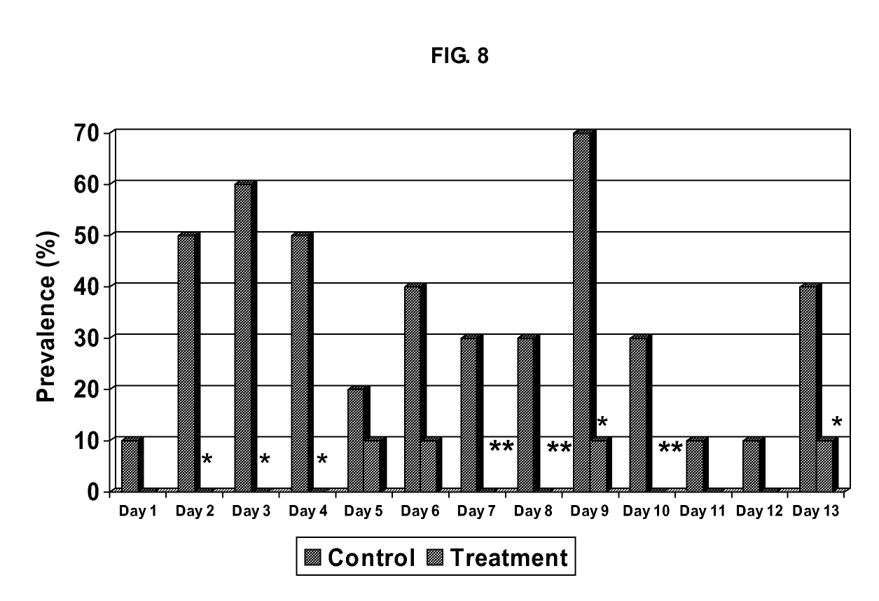

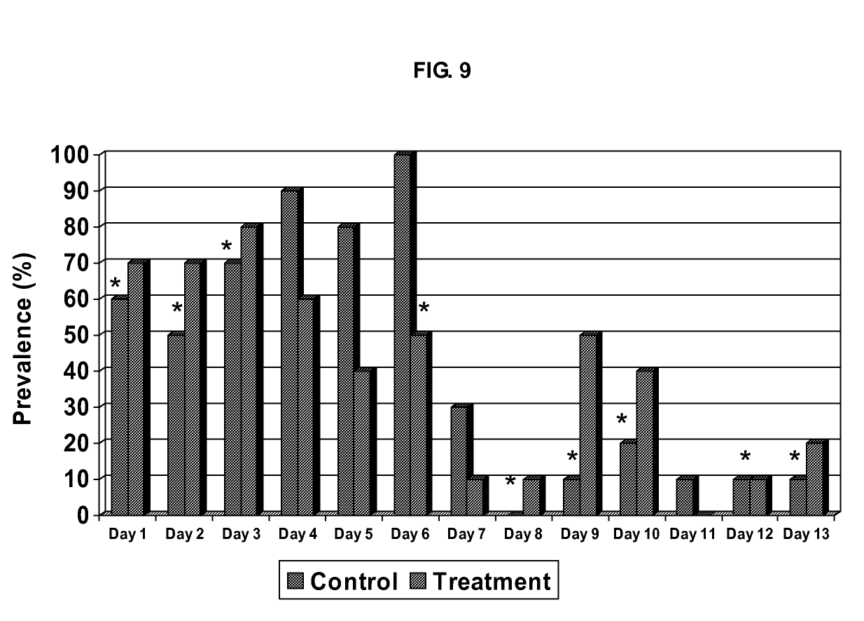

Results showed that use of Tasker Blue® in the scalder and in a post-pick dip solution had a significant (p<0.05) impact on APC (see e.g., FIG. 4, FIG. 5) and E. coli (see e.g., FIG. 6, FIG. 7) counts on chicken carcasses. Similarly, use of Tasker Blue® in the scalder significantly impacted Salmonella prevalence values (see e.g., FIG. 8). Such reduction can, later in the process, impact the Salmonella load coming out of the chillers. Lesser Salmonella reductions post-pick and post-dip (as compared to post-scald reductions) may be explained by possible cross-contamination by the pickers (see e.g., FIG. 9).

These data indicate that scalder disinfectant provides an effective means of lowering total numbers of bacteria, enteric bacteria (E. coli) and Salmonella in particular. The high heat and organic load of the scalder currently precludes the use of other products. Thus, use of a disinfectant, such as Tasker Blue®, in the scalder and/or as a post-pick dip solution can assist processors in meeting the USDA-FSIS Salmonella Performance Standard.

### Example 2

The effect of decreased scalder temperature in combination with the use of a scalder disinfectant on yield was examined.

Routine problems encountered with lowered scalder temperature include increased Salmonella prevalence and Poor picking and epidermis removal. Salmonella's maximum growth temperature is 113° F. Practitioners in the art generally recommend a minimum of 10° F. above the maximum growth temperature to prevent growth in the scalder. As such, it is routine in the industry to the maintain the scalder at a minimum temperature of 123° F. In a preliminary study where a scalder was maintained at 114° F., Salmonella were observed at a level of 10⁵ (100,000)/ml of scalder water, resulting in every carcass run through the scalder being inoculated with Salmonella during scalding.

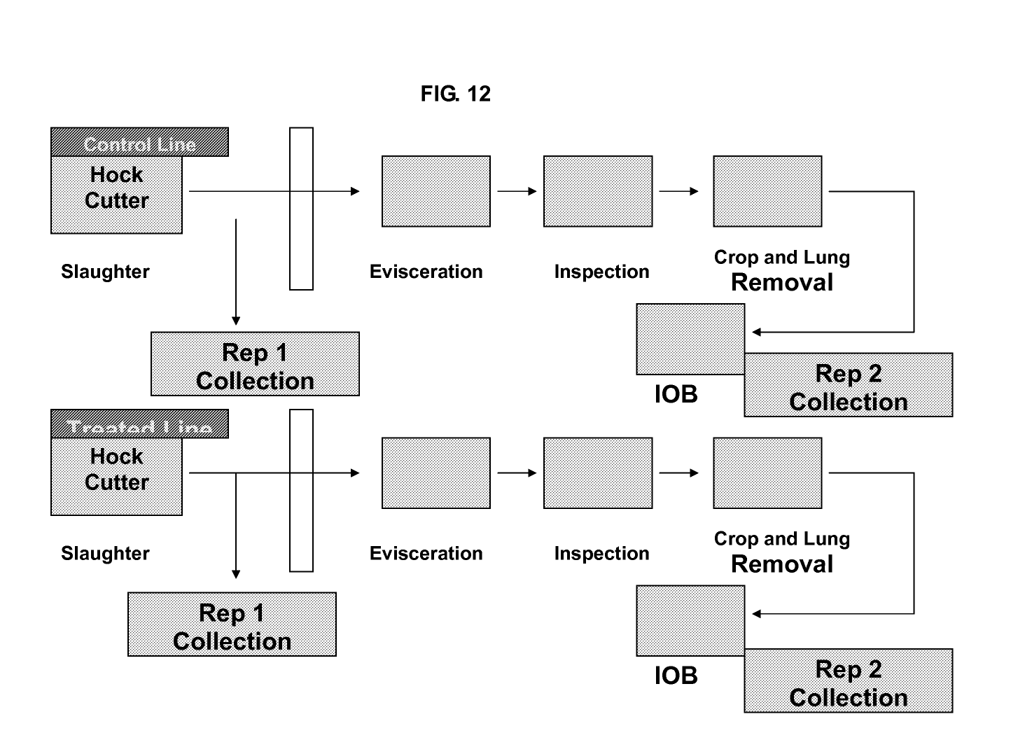

Collection procedures were as described in Example 1, except as otherwise noted. Control scalder temperatures were 132°, 134°, and 136° F. for the first, second, and third scalder tanks, respectively. Scalder temperatures the disinfectant treated tanks for carcasses collected post-hoc cutter were 113°, 123°, and 138° F. for the first, second, and third scalder tanks, respectively. Scalder temperatures for the disinfectant treated tanks for carcasses collected pre-IOBW were 110°, 114°, and 134° F. for the first, second, and third scalder tanks, respectively. 100 carcasses from the same flock were collected post-hock cut and divided between a treated line and a control line (see FIG. 12). 100 carcasses from the same flock were collected pre-pre inside outside bird washing (IOBW) and divided between a treated line and a control line (see FIG. 12). Carcasses were weighed and scalder water assayed for Salmonella. Three replicate trials were conducted for a total of 1200 carcasses over 6 flocks.

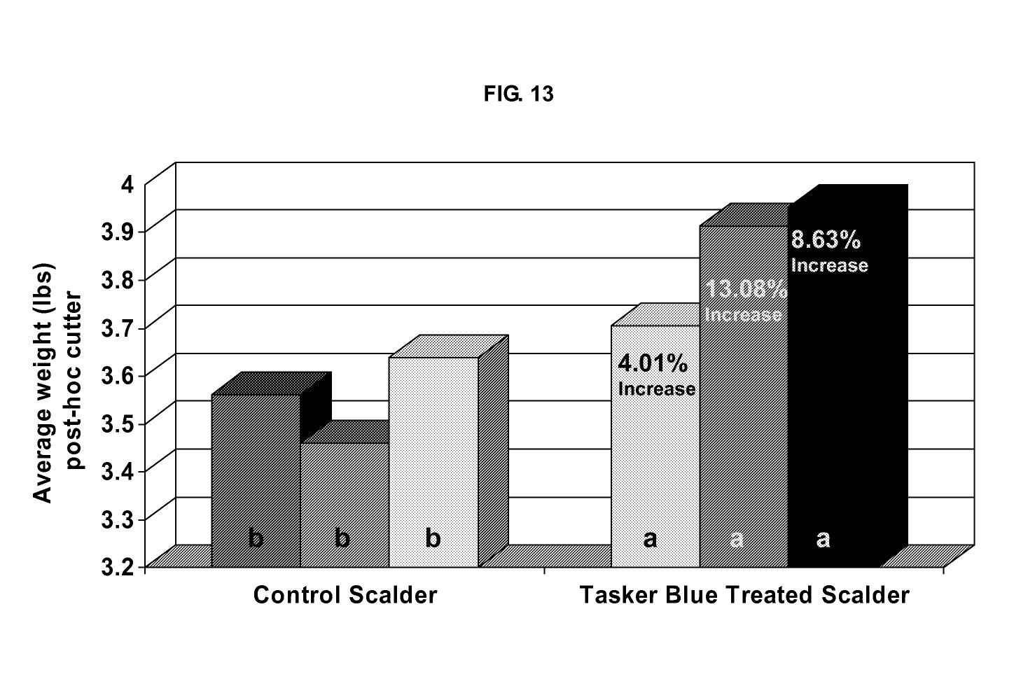

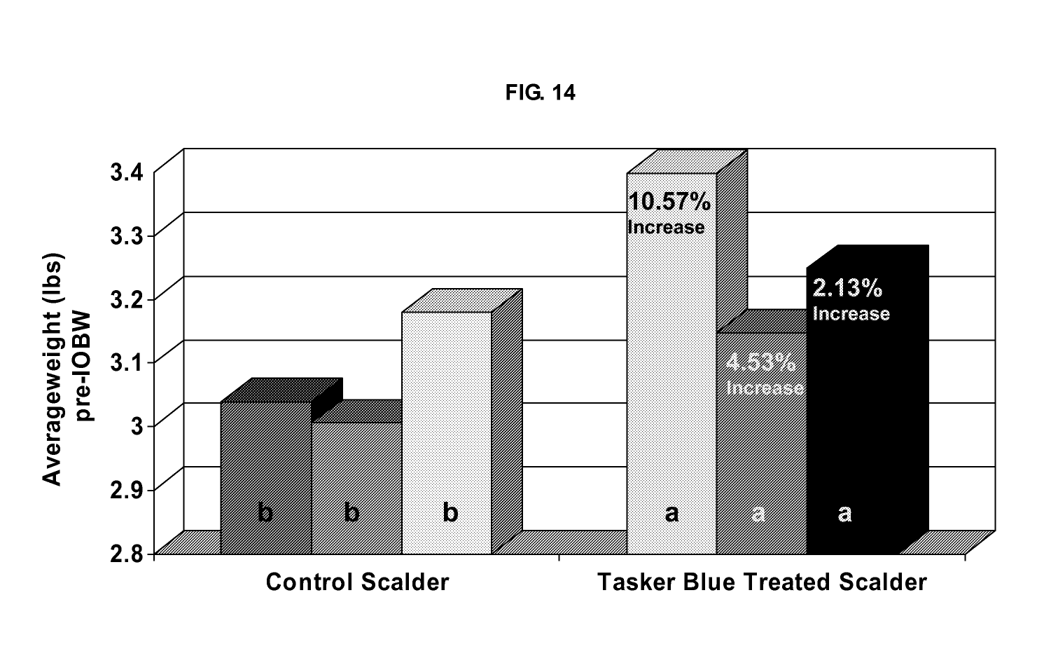

Results showed reduction of scalder temperatures for post-hock cut (see e.g., FIG. 13) and pre-IOBW (see e.g., FIG. 14) as compared to controls resulted in significant increases (p<0.0001) in weights of the individual carcasses. Over 6 repetitions, there was an average yield increase of 7.14% when low scalder temperatures were used. Such yield increase, when applied to the plant wherein the tests were run, would result in a monetary gain of approximately $38,000 per day for that processor. Thus, running the scalders at lower temperatures (110 to 113 on Scalder 1; 114 to 123 for Scalder 2, and 134 to 138 for Scalder 3) produces a significant increase in yield. Furthermore, no appreciable levels of Salmonella were found in the scalder water treated with Tasker Blue®. Also, feather removal was similar for both lines.

Thus, a scalder disinfectant, such as Tasker Blue®, is an effective means of reducing bacterial populations in scalder water and on carcasses during scalding. Applying a scalder disinfectant, such as Tasker Blue®, as a post-pick dip reduced cross-contamination observed during picking. Disinfection of the scalder water and use of acid allows scalder temperatures to be reduced significantly. And reduction of scalder temperatures resulted in quantifiable and significant increases in carcass yield.

### Example 3

The effect of disinfectant added during scalding, spraying, and chilling on Escherichia coli counts and Salmonella prevalence was examined.

Thirty broiler chicken carcasses were collected prior to scalding in a commercial processing facility and transported to a small-scale poultry processing plant. Collection procedures were as described in Example 1, except as otherwise noted. These carcasses were inoculated with a naladixic acid resistant strain of Salmonella typhimurium (obtained from the Poultry Microbiological Safety Unit at the USDA-Agricultural Research Service's Russell Research Center) and were allowed to attach to the carcasses for 3 hours. Fifteen control carcasses were scalded in commercial scald water, sprayed with tap water, and chilled in tap water for 1 hour as controls. Fifteen test carcasses were scalded in commercial scald water containing Tasker BLUE® at 2 ppm copper, sprayed with a 2 ppm copper solution of Tasker BLUE®, and chilled in a concentration of 2 ppm copper Tasker BLUE® for 1 hour. After treatment, carcasses were rinsed using the whole carcass rinse procedure. Half of each rinsate was placed on blue ice and shipped to a commercial research laboratory (ABC Research Corporation, Florida) for E. coli testing. The other half was evaluated for Salmonella prevalence by direct plating onto brilliant green sulfa plates containing 200 ppm naladixic acid. Plates with characteristic bright pink colonies indicated that the carcass was positive for Salmonella. The bacteria were removed from the plates and confirmed using serological testing.

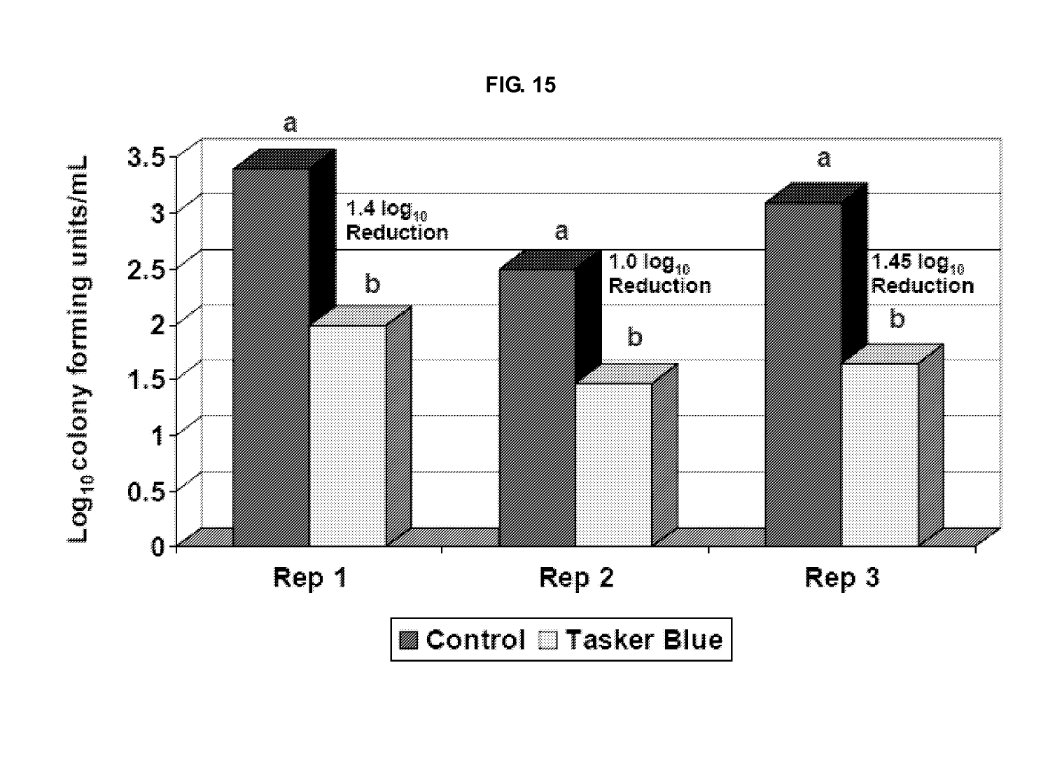

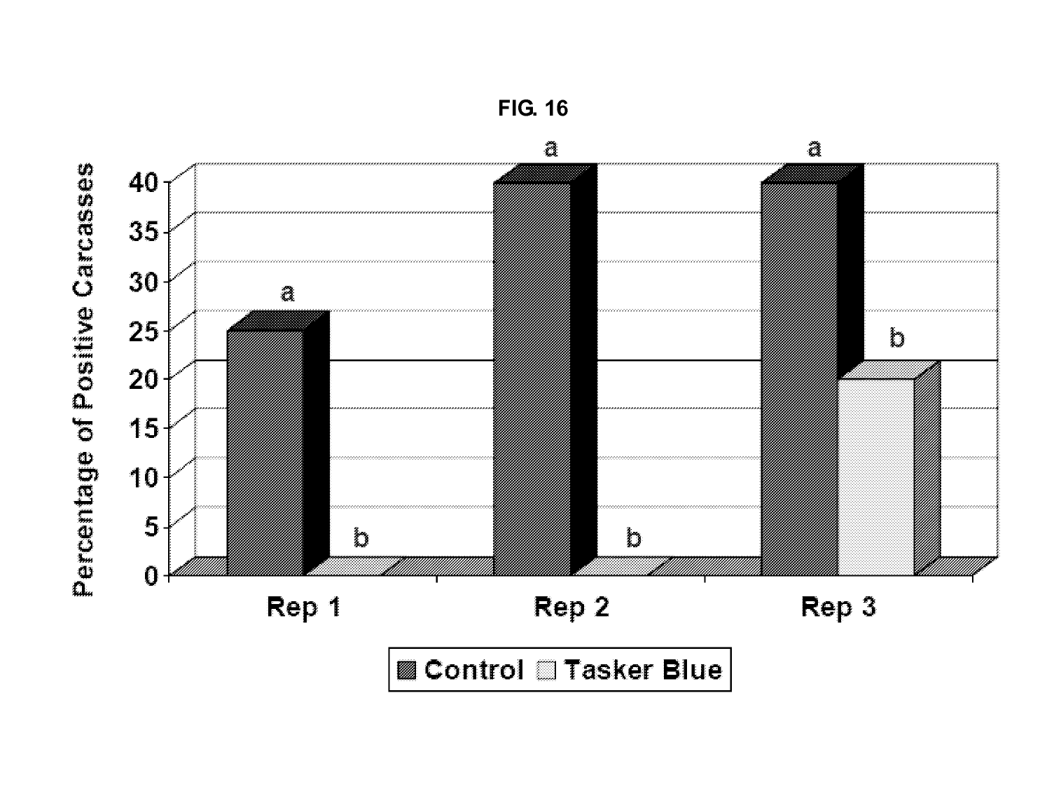

Results showed that scalding, spraying, and chilling broiler carcasses with Tasker BLUE® significantly decreased E. coli counts. For example, FIG. 15 shows significant Log10 reductions (p<0.05) in E. coli of 1.4, 1.0, and 1.45 in Reps 1 to 3, respectively. Thus, BLUE® can lower E. coli on chicken carcasses to the USDA acceptable level of <100 cfu/mL. Results also showed that scalding, spraying, and chilling carcasses in BLUE® lowered Salmonella typhimurium prevalence on chicken carcasses (see e.g., (FIG. 16)

It is noted that reductions of this magnitude rarely, if ever, occur when using trisodium phosphate (TSP) or acidulated sodium chlorite (ASC). Overall, these data indicate that using BLUE® in the scalder and/or in combination with applying it using a sprayer and/or during immersion chilling is effective for reducing populations of E. coli and Salmonella prevalence.

### Example 4

A study was conducted to determine if any of the primary ingredients in pHarlo Blue® 0020 (ammonium sulfate, sulfuric acid, copper sulfate) evolve into the air when introduced in a commercial poultry scalder. A diagram of the process including treatment points and sampling points is depicted in FIG. 4:1.

Air samples were collected using an impingement system as depicted in FIG. 4:2. The air was pulled, using a vacuum pump (Welch Dryfast Ultra Vacuum Pump), at a rate of 1.2 L/minute through 100 mL of deionized water for 5 minutes. The total amount of air evaluated was 6.0 L for each sample. This was conducted over a two hour period at the Pilgrim's Pride poultry processing plant in Athens, Ga. For the controls, air was sampled above the scalder without the addition of any pHarlo Blue® 0020. A total of 10 samples were collected for the controls. The scalder was dosed with pHarlo Blue® 0020 until a pH of 2.2 was achieved. A total of 6 air samples were collected for the treated scalder water. All samples were collected into a glass sample jar and decanted into sterile Nalgene bottles. Immediately after collection, all samples were placed on ice and sent via overnight express to Enthalpy Analytical Laboratories for chemical analysis.

For ammonium sulfate, samples were evaluated by testing the water for the presence of ammonia according to the procedures outlined in EPA CTM 027. Analysis was performed using a Waters 430 conductivity detector attached to a Hewlett-Packard series 1100 High Performance Liquid Chromatograph. A calibration curve was analyzed prior to the samples yielding a suitable correlation of 0.99930. Ammonia eluted at approximately 3.0 minutes, separated well, and was easily identified. Ammonium sulfate was determined by calculation from the ammonia data.

For sulfuric acid, samples were evaluated by testing the water for the presence of sulfate using a Waters 430 conductivity detector attached to a Hewlett-Packard series 1100 High Performance Liquid Chromatograph in combination with an Alltech ERIS 1000HP Autosuppressor. Separation was accomplished by a Dionex IonPac AS14A 250×4.0 mm analytical column using 8.0 mM Na₂CO₃/1.0 mM NaHCO₃ as the eluent at 1.2 mL per minute. A calibration curve was analyzed prior to the samples yielding a suitable correlation of 0.99922. Sulfate eluted at approximately 9.6 minutes, separated well, and was easily identified. Sulfuric acid was determined by calculation from the sulfate data.

For copper sulfate, samples were evaluated by testing the water for the presence of copper using a Perkin Elmer ELAN 6100 ICP-MS. Copper was converted to copper sulfate by multiplication of the copper data by the molecular weight of copper sulfate pentahydrate, divided by the molecular weight of copper.

All sample numbers were encoded to prevent identification during analysis. The data were subjected to a two-tailed t-test using the Statistic Analytical Software (SAS) program.

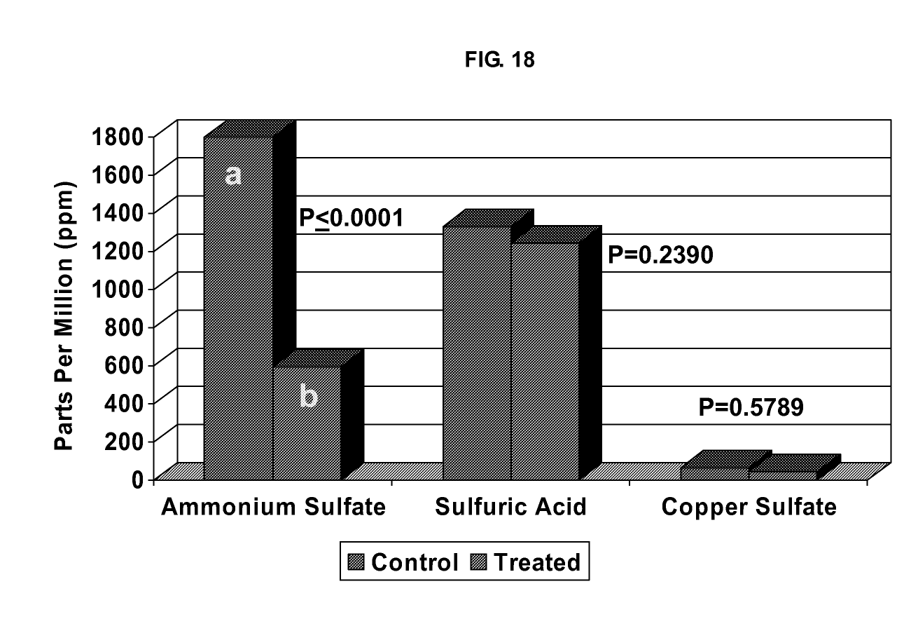

Results showed that the levels of the three main ingredients in pHarlo Blue® 0020 (ammonium sulfate, sulfuric acid, and copper sulfate) were not elevated in the air sampled above the scalder when the scald tank was dosed with pHarlo Blue® 0020 (see e.g., FIG. 18). In fact, ammonium sulfate was significantly higher in the air above the untreated scalder water. No significant differences (p<0.05) were observed for sulfuric acid or copper sulfate levels in the air above the untreated (control) and treated scalder water. From these results, it is apparent that dosing the scalder with pHarlo Blue® 0020 resulted in no significant increase in the levels of any of the chemical components in the air directly above the scalder. Further, in multiple pilot scale studies, along with the study described herein, none of the personnel working around the scalder described having any adverse affects associated with breathing the air around the scalder after dosing it with pHarlo Blue® 0020. Thus, emission of any of the components of pHarlo Blue® 0020 into the air during scalding is inconsequential.

### Example 5

The inhibitory activity of acidic buffered copper-containing disinfection agents was determined against Escherichia coli ATCC 11229. The disinfection agent was commercially available Tasker Blue® (sulfuric acid, ammonium sulfate, copper sulfate pentahydrate).

Test samples were prepared for testing at pH levels of 2.0, 2.5, 3.0, 3.5, and 4.0 in combination with copper concentrations of 0 ppm, 1 ppm, 2 ppm, and 3 ppm. Tryptic soy Broth was prepared half strength as a standard inoculum of 0.5 McFarland. The test sample was added to a sterile tube, along with the same amount of standardized Escherichia coli ATCC 11229 inoculum. The pH of the sample was recorded and adjusted as indicated on the test sample bottle. Tubes were incubated for 24 hours at 35° C. and the inhibitory concentration was determined as the lowest concentration showing visible inhibition of the growth of the organism. All samples were run in duplicate along with positive and negative growth controls. Final pH of test samples were recorded following completion of 24 hour incubation.

Results showed that complete inhibition of microbial growth was achieved with all solutions except the following solutions, in which microbial growth was detected: pH 4.0 Cu 0 ppm; pH 4.0 Cu 1 ppm; pH 4.0 Cu 2 ppm; pH 4.0 Cu 3 ppm.

### Example 6

The inhibitory activity of acidic buffered disinfection agents on aerobic plate count (APC).

Five formulations were tested.

Mark I: a 24 hour high temperature reaction process at approximately 300-350° F. with a stabilization step after overnight cooling. Composed of reacting 98% sulfuric acid with a 26-28% by weight ammonium sulfate in water solution. The order of addition was ammonium sulfate solution to sulfuric acid. Electrolysis of the reacting solution was applied for 1 hour at the start of the process. The stabilization step was the addition of more ammonium sulfate solution to ensure that the reaction is complete. The Tasker Clear product formed was a buffered acid solution of a strong acid (sulfuric acid) and a salt (ammonium sulfate) of a strong acid and strong base.

Mark II: a 2 hour low temperature reaction process at approximately 200-210° F. with a stabilization step immediately after the 1 hour electrolysis period. This was the same process as in the Mark I product above except that it was performed at a lower temperature and a shorter period of time. The ingredient amounts were adjusted to account for no lost of water as was seen in the Mark I process. The Tasker Clear product formed was a buffered acid solution of a strong acid (sulfuric acid) and a salt (ammonium sulfate) of a strong acid and strong base.

Mark III: a low temperature reaction process in which the 98% sulfuric acid was added slowly to a 30% by weight ammonium sulfate solution. The addition was done continuously until all the ammonium sulfate solution was added. There was no stabilization step. The addition order was the reverse of the Mark I, II, IV, and V processes. The temperature was maintained in the 150-200° F. range during the addition process. No electrolysis was performed during this process and hence the name ‘cold process’ was given to it. The Tasker Clear product formed was a buffered acid solution of a strong acid (sulfuric acid) and a salt (ammonium sulfate) of a strong acid and strong base.

Mark IV: a 4 hour high temperature reaction process at approximately 300-350° F. with a stabilization step after cooling. Composed of reacting 98% sulfuric acid with a 26-28% by weight sodium sulfate in water solution. The order of addition was sodium sulfate solution to sulfuric acid. Electrolysis of the reacting solution was applied for 1 hour at the start of the process. The stabilization step was the addition of more sodium sulfate solution to ensure that the reaction is complete. The Tasker Clear product formed was a buffered acid solution of a strong acid (sulfuric acid) and a salt (sodium sulfate) of a strong acid and strong base. (Note: In this process sodium sulfate was substituted for ammonium sulfate.)

Mark V: a 4 hour high temperature reaction process at approximately 300-350° F. with a stabilization step after cooling. Composed of reacting 98% sulfuric acid with a 26-28% by weight sodium sulfate in water solution. The order of addition was sodium sulfate solution to sulfuric acid. There was no electrolysis during this process (cold process). The stabilization step was the addition of more sodium sulfate solution to ensure that the reaction was complete. The Tasker Clear product formed was a buffered acid solution of a strong acid (sulfuric acid) and a salt (sodium sulfate) of a strong acid and strong base. (Note: In this process sodium sulfate was substituted for ammonium sulfate, and no electrolysis was performed.)

Results showed that all formulations exponentially reduced the aerobic plate count (see e.g., Table 2).

TABLE 2

| | | Log₁₀ | Log | | | Log₁₀ | Log |
|---|---|---|---|---|---|---|---|
| Time | Counts cfu/ml | cfu/ml | Reduction | Time | Counts cfu/ml | cfu/ml | Reduction |
| | Butterfield Buffer | | | | | | |
| | Control | | | | DI Water Control | | |
| 0 | 845 | 2.93 | | 0 | 1015 | 3.01 | |
| 5 | 780 | 2.89 | | 5 | 1075 | 3.03 | |
| 15 | 785 | 2.89 | | 15 | 940 | 2.97 | |
| | | Ave = 2.90 | | | | Ave = 3.00 | |
| | Mark I Solution | | | | Mark II Solution | | |
| 0 | 140 | 2.15 | 0.85 | 0 | 100 | 2.00 | 1.00 |
| 5 | 25 | 1.40 | 1.60 | 5 | 30 | 1.48 | 1.52 |
| 15 | 5 | 0.70 | 2.30 | 15 | 0 | 0.00 | 3.00 |
| | Mark III Solution | | | | Mark IV Solution | | |
| 0 | 65 | 1.81 | 1.19 | 0 | 110 | 2.04 | 0.96 |
| 5 | 0 | 0.00 | 3.00 | 5 | 40 | 1.60 | 1.40 |
| 15 | 0 | 0.00 | 3.00 | 15 | 0 | 0.00 | 3.00 |

| | Mark V Solution | | |
|---|---|---|---|
| 0 | 125 | 2.10 | 0.90 |
| 5 | 20 | 1.30 | 1.70 |
| 15 | 5 | 0.70 | 2.30 |

NOTES:
* Log Reduction based on DI Water average log₁₀ = 3.00
** Counts are the average of duplicate APC plates

What is claimed is:

1. A method of reducing a microbial population on an animal carcass during processing comprising the steps of:
a) scalding an animal carcass;
b) removing feathers, hair, or hide from the scalded carcass;
c) eviscerating the defeathered, dehaired, or dehided carcass;
d) washing the eviscerated carcass;
e) chilling the eviscerated carcass; and
f) applying to the carcass during processing a disinfection composition comprising an acid and a buffer, in an amount and time sufficient to reduce a microbial population;
wherein applying the disinfection composition (f) is performed during or immediately after at least one of steps a, b, c, d, and e.

2. The method of claim 1 further comprising the steps of:
(g) recovering at least a portion of the applied disinfection composition;
(h) adding to the recovered composition a sufficient amount of disinfection composition to yield a recycled disinfection composition; and
(i) applying the recycled composition to a carcass during processing;
wherein applying the recycled disinfection composition (i) is performed during or immediately after at least one of steps a, b, c, d, and e.

3. The method of claim 1 further comprising the step of applying the disinfection composition to an animal prior to or immediately after it is sacrificed to form an animal carcass.

4. The method of claim 1 wherein applying the disinfection composition comprises submersing, rinsing, or spraying the carcass in or with the disinfection composition.

5. The method of claim 2 wherein applying the disinfection composition or the recycled composition comprises submersing, rinsing, or spraying the carcass in or with the disinfection composition or the recycled composition.

6. The method of claim 1 wherein applying the disinfection composition is performed at least by (1) submersing the carcass during scalding and (2) rinsing, spraying, or submersing the carcass after scalding but before eviscerating.

7. The method of claim 6 wherein rinsing, spraying, or submersing the carcass after scalding but before eviscerating is performed at a sanitization station, wherein the sanitization station is intermittently fluidly connected to an apparatus for removing feathers, hair, or hide; an apparatus for scalding, or both an apparatus for removing feathers, hair, or hide and an apparatus for scalding; so as to allow transfer of recycled composition from the sanitization station to the feather, hair, or hide removal apparatus, the scalder apparatus, or both the feather, hair, or hide removal apparatus and the scalder apparatus.

8. The method of claim 1 wherein applying the disinfection composition is performed at least by (1) submersing the carcass during scalding; (2) rinsing, spraying, or submersing the carcass after scalding and before eviscerating; and (3) submersing the carcass during chilling.

9. The method of claim 8 wherein rinsing, spraying, or submersing the carcass after scalding but before eviscerating is performed at a sanitization station, wherein the sanitization station is intermittently fluidly connected to an apparatus for removing feathers, hair, or hide; an apparatus for scalding, or both an apparatus for removing feathers, hair, or hide and an apparatus for scalding; so as to allow transfer of recycled composition from the sanitization station to the feather, hair, or hide removal apparatus, the scalder apparatus, or both the feather, hair, or hide removal apparatus and the scalder apparatus.

10. The method of claim 1 wherein application of the disinfection composition occurs during submersion scalding, wherein scalding occurs at a reduced temperature so as to increase post-scalding carcass yield.

11. The method of claim 10 wherein scalding in at least one scalding tank occurs at a temperature of about 110° F. to less than 123° F.

12. The method of claim 11 wherein scalding the animal carcass occurs in at least three scalding tanks; a first scalding tank operated at about 110° F. to about 120° F.; a second scalding tank operated at about 110° F. to about 125° F.; and a third scalding tank operated at about 120° F. to about 140° F.

13. The method of claim 1 wherein the disinfection composition is of a pH of about 1.5 to about 4.

14. The method of claim 13 wherein the disinfection composition is of a pH of about 2 to about 3.

15. The method of claim 1 wherein the disinfection composition comprises (i) sulfuric acid and (ii) ammonium sulfate or sodium sulfate.

16. The method of claim 1 wherein the disinfection composition further comprises an antimicrobial metal.

17. The method of claim 16 wherein the antimicrobial metal is selected from the group consisting of copper, zinc, magnesium, silver, and iron.

18. The method of claim 17 wherein antimicrobial metal is copper in an active ionic form.

19. The method of claim 18 wherein the disinfection composition has a copper concentration of about 1 ppm to about 20 ppm or is added to carcass processing water in an amount sufficient to provide a copper concentration of about 1 ppm to about 20 ppm.

20. The method of claim 19 wherein the disinfection composition has a copper concentration of about 2 ppm to about 4 ppm or is added to carcass processing water in an amount sufficient to provide a copper concentration of about 2 ppm to about 4 ppm.

21. The method of claim 20 wherein the disinfection composition has a copper concentration of about 3 ppm or is added to carcass processing water in an amount sufficient to provide a copper concentration of about 3 ppm.

22. The method of claim 1 wherein the disinfection composition comprises (i) sulfuric acid, (ii) ammonium sulfate or sodium sulfate, and (iii) copper sulfate.

23. The method of claim 22 wherein the disinfection composition comprises sulfuric acid, ammonium sulfate, and copper sulfate.

24. The method of claim 23 wherein the disinfection composition comprises about 98% to about 99% water; about 0.1% to about 0.5% copper sulfate; about 0.1% to about 0.5% sulfuric acid; and about 0.1% to about 0.5% ammonium sulfate; and the disinfection composition has a pH of about 2 to about 3; a specific gravity at 25° C. of about 1.002; a boiling point of about 212° F.; and a freezing point of about 32° F.

25. The method of claim 1 wherein the disinfection composition further comprises a stabilizing agent, wetting agent, hydrotrope, thickener, foaming agent, acidifier, pigment, dye, surfactant, or a combination thereof.

26. The method of claim 1 wherein the carcass comprises poultry, red meat, or pork.

27. The method of claim 26 wherein the carcass is a poultry carcass.

28. A carcass processing system comprising:
a scalding station;
a feather, hair, or hide removal station; and
a sanitization station;
wherein each station is intermittently fluidly connected via a buffered acidic disinfection composition.

29. The carcass processing system of claim 28 wherein the carcass comprises poultry, red meat, or pork.

30. The carcass processing system of claim 28 wherein the disinfection composition is of a pH of about 1.5 to about 4.

31. The carcass processing system of claim 28 wherein the disinfection composition comprises (i) sulfuric acid, (ii) ammonium sulfate or sodium sulfate, and (iii) an antimicrobial metal.

32. The carcass processing system of claim 31 wherein the antimicrobial metal is copper in an active ionic form and the disinfection composition has a copper concentration of about 1 ppm to about 20 ppm or is added to carcass processing water in an amount sufficient to provide a copper concentration of about 1 ppm to about 20 ppm.

33. The carcass processing system of claim 28 comprising at least three scalding tanks, wherein a first scalding tank is operated at about 110° F. to about 120° F.; a second scalding tank is operated at about 110° F. to about 125° F.; and a third scalding tank is operated at about 120° F. to about 140° F.
